SEQUENCE LISTING (9 sequences)

SEQ ID NO: 1 - DNA, 13686 bp, unidentified
  source 1..13686: mol_type = unassigned DNA
ctattcgtttccacaaacttcattaagataatcattccatttctcttttataaggttaggcttgaatctctttaaaacacctcttgctttatttccaatgctatctgctactagtggattttgaaatagataaatcaatttttccgctaacattgaagcatcacctctattcacgataaacccgtttactccattttctataagaaatcttgcacctccaggactcgtatcagttgttattactggaagacctattgacatagcttctaaaagagcattagaaatgccttcaaaatccgatgacatcacgaaacattttgaagtagctatatattgagccggattattaactttccctttaaactcgaccttatcagaaatgcctaaattcttggtcaaacgcatcatttctgataaatcttcaccatcaccataaaggcataatttaacattatctatagttttgcaaaaaaatgagaatgctatcaacagtacatctattctcttttgttgcacatgtatcctcccaacataagaaatctcatctttcctttgagaaaaaggcaaaatcgtttgaatccccgtaacaaatatcggatttggaatgacagatgttttctttttaatgatattcttaaagaacgattgggcttgaggtagttgaaagacagcaccatcagcccatctataaaaaaattgagctattttagcattcagtggcaacttagcataaggatcactcctttcacaaacaactaccggcacatgagtttggatagcagcaaatatactatacaatcctgcatcatataagaaagcaacaattacatcaggatggatttcttttatcttcttccgtaaatcaaatataattttccatcgttcggctttctcccttattacttcgctccattcaacgttctcagaaaatgtatattctgtatttgaataatagaaagtatgcagaaatatacgattctcgttatattcagataaactatttaccgtccaattaaacatttttgagtacccatcataagtgactctttgcccgacaaacaaaatccttttattcataattatcgcttataaaatctaataatctatgcacatattttacattattaaaatgcttttggaatatggcatgacctttttggcctatttcagtagcatgagatttattttgcataattttatcaaacaaaaaagctaattcatttacatccaattcgtttataataaaagcagagtttgtatcaaaataataaggaacttctccaacacgatttgttattatgggtcttccagttgaggtatattctgctattttctgactaaatcgtgctatgtcttgtagattggaagcatctaaaggaatcaataaagctaatgattcagaatacaacttcaataaatcactataaggcaacccttgtttgatagaaaccttttctcccaatccgtttatttctatctgttcttggattttttgtatatcgttttgatttccagaaagaaccaacaccaaatttgcattcccttttttagaataaatatcatacgcatctagaataaaatcaataatgcgtttatatgcagcacttccgcaatatgtgaactgttcccgaattatgtctactacagaattttcatttgtagcagacaagactggagtcatcaacataggtttcttgtatttttcacatctatttttcaaaaaatgagaaataggcaaaatgaaatcacaaaaatatccgaaataacgatcgaatgcctttgtgctatataaagtatacttagatggattttgtaatgttgtatatcgtaattcatgatacaggactccaattttatatcccaatattttcatccaaatacattctattatgtaaataataaaatgattggaagtcagaatggtatatttatattctttcctaaggcgaaataataatattaaaatctttacaagattgtacagcctacccaaaagacctttttgaggatatatgccatataatatccccgcatttttaccatgaatctctttttcaatcttattatagccccacataggattaatgatatacaccttccgaccaaagtgcagcaaaccttgtgataagatttctactttggtgtttcctgcagaatattcaaatggataatcttgtgtggagtctaggaaaatatagttagctctcactttattttgtcttttaaataataatagatgttgcagataagataatataatcgaatcttagcgtacaaaaaacagtcatatttattaattctcatattaagaccatttattttttccaataatcctaaccgcttaatttcgttatggtaatgtgaaacaactttcaatagaatacggagattaacttccaaatagtctcgcttaaaatctaaattttggcttaagacgtattctaacaatcctatgtaaaatactacatatccatacaaaaaacgagcattagtggagttttttaaattggtaagtcgatcgtcagctttatcatcacaatatatcgtatgaacctctggcaatacagagactattatacttttctgaattaagtatctaaataccaagtcaacatcctcataactccacatggattcattatatccattaacattcaaaagaggttccttggtaaacatccaagcctgtgtacctacaaaagcaccatcttgatgcagcaagaaactatgcaaacaagatttctccaatatcggagaaggcacaacatctatattccttcttttgtaatgaagtcccgcaacacacaaatcactacccttgtctatcattataccaatctgactctcaatcctatgtggaagcatgatatcatccgaatcaaaaaagcaaatatacgaacccttactatgctttaaaccatagtttcttgccgaagaaggccctttattactttggcataagtatacaattctgtcatcgttgtaatcacgaattaaagcttccaatagtgcggaaacatttttagaagaaccatcatttacaatgatgatttctaaatttctataagtttgacctacaatactgtctatagtaggtttaatattagccctgttataaactggaattatcactgatactaatggcactctcatttttcaaatatttaacaccttatacggaacgttgtgtatcaagcttgcgcgttttgcaaatccggaattatacataatgcttgttttgactaaatatattttcttagccttagccagcataaataagtcaagaaaggattttatataaggtaaatcttgtgatactgtatggtagtccatgtgatatacccttccttctatgatagaaacgtaatcaatatcctttacatactccaaaaaggtaatgctatctgatgttatgagaattttttcatcaggattctctttatgcaacatgttgattgcatttttgcactgttccatcaagttttgccgcttctcttgttttaatactgggtaattaccttctttaaaatcccccaataattgttggaatctataaaccattgcaatatactctcctcctattttctcataattttcactcaataaattcgagagtttgtctgttggtttgaaaagttcatgaaacaaccgggaaaagcatttgtctcccaaatctatattggtataaatatggttctgtgcatctcgtacaacacgctttataagatacctttcttgaaaagcatgttcaaatggtaaatctctattatatatatatattacatttgtattctgagaaaagtccattttagaatcatcaatcaaccaatcatatttattgggcatcaggtaatcttctattctaaatggagacacaaaataaatcttaaaagaaactttcattttctttgctatataatacaatgacacacaacctcttaacctgtcagaaagaccaccatgaagcatagagccgtcagctatatacactatgccttggaattgcttagaacaagttggagcttgatatctatgccgaagtacagcctcaacagcagtgttcattattttaccaacgtataaaaaatattttagccctcgcagataatctatcatacttacttactgatttatagtcttataaaaataatttttaatttcttgatcagacattttataaaggtctgtattatatgttaaaccaagaaccatcggaaatacaaaatagcttggtgtgaatgcgaatgaatattctatccataacatcaaccacataaatggtactaccaatcccactatcttacagaatttattattagatacaactatacaatttatactcatcttatacaacattaacatgtaaaagataagcccaaaagttccaaaccaagtaaaaatggactctatatgcacctcgctaattctctccatcgaggttccttttaaaaaaaatccgagtgacgacatgcggtctatttgaaataaagactcatgtccataatagggagtgcgcccccaaataacatatccattattaaaggcagactctatggcctctaaataaataaaagttctactatcctctaaggcatttgctgttctatcattagtttcgtcataatattcaaatccttctcccatataagatttcatatcaaatacgttaaaatcataacgtaaagccaaaattaaagctaacaatggtatacaaaagacaaacgtgtgagttatcgcaaataatctccttgaaagaaacgggacataaaacatcatcaatagcataatacagaagaaatatctcatatagtatatgcgctgttctgttgttaaaatcgataaaataaacactaaaatcactatgaacttacgtttcgtattcaggtagggatataataaaaagaaaaataaattcattcctagtatatctgcataattaagatataatgcaaatggaagtaacaacacaaagaacggtataatatacttaaaatatagacttgaaacccctctcaagaagttttgccttccataaaatattatgcccaataatagagacaaggaaaacagagcatctgcctgatttatcggaaacaaagaagaaggatgaatgcaaagtttggcaaagcctactactaaataggcaatgatataatataaaaagtaattggtaaattggttctgtttaatatgcagattcttttttattaagaaaacaatagacattataattgtcatagtccaagatgcaaagatctgcaatctttgaggatatttgaagctttccacaattcttgtaacatacaataaaaaaaacactgcaacgataaccgtaaaatattttattatataattcgttaccaataatagaaaggtgtttttattaataagtttcataagttatctatttttttaataaatctggcaggtacacctccccaaatctcgtatgggggaatatctttggttacgagagaacctgccccaataatagaattattaaatattcttaccccccccattattatagaattcatgcctatccatacattatcatctattattataggacgcgtaactttagcatatctgtcttcatggtcatttctgtcacctatgataacatttgccccacaaaggacattgttgccgattaacacactcttgtcgcaaacaatactaacagacgagaatccacaatcatttcctatttgtattatagccccagacttccctgtttgaatgatgcaaggattacgcagccctctaaaattgaataaggaggacgaattaaatctacaacgtttaccaattgttatattactattgcgcgttttataaaaaataattcgacctcggaagctacatcctttatcaagatagataccccaaacaaatgcttttacttttgctattacagtataatgtttttttataaatttatttcgataccaatacagtttttcaatgattgagctcataatatattttttttgagattgttttatataaatgattcgtctggttaatcaagtgattgatgaaaatgaatctgagaagcctatcaacacatatcagtccaataaatgctacgaatgaaaaaaagaccatcagtataatgaacatatatcctggataatgagtagcagaaaacaacgcgtaaatttgctcaacaaggtctaccaaagcctttcttgcatatacatcctcatcatgcatgagatatacggcgaatgttgatgccgatatattatttaggaaactgctttttatatttattcctttgaaaaacaggaaaaccaaaaccgattgtaatacgaccaaaggattgatatatcctcgaaagctccccaaataatgtctttctaaaaagattagaatagaaacaatcaagatactgtaaatacactttttcctatttacattaaaatcaaaatatctaatacttccacctatgaaatacatatagacaaagtggttgtaattaaatccattcaaattataatcactatgtaagattcctccacatactatattcaaaaaagttaatagacataaaaatagaatcacattctttctatttgcagagaaagccatgtttaatataggagcggtaacgaacaaatagaaatatatttcaacaaaccaccacggactatttctataaaagaaatggaagcgctttaaaaatgccataaaagacagtgaagtctcagagaataaggaaaaaacaatccatattgcaacagaataaaatgcacaagtaataaaaaatcggaaaaaactgtttgcttttaatttgattccaaaatatccagatatgagaagaaaggcattaacgtgaaatcctaagaaactctgtatacccattgaaaagttattggcacaattatcaagcaaaagtcgatcgcatccgtgatacaaaatgtggctacataggattgaaaatatgcagataatcctcagtaattcaaaatttgaaagccggttatttcccgatatagacatattacttatgtttaattatagatttatatagataatctctttctgatttagttaaccctagaaatagaatgacgtttgttaatattacgaatgaaataaccgcacatacgaaatcataggttattgtttgatttatatgtgttaaactgagatgatacagggttatcataaaaatggaactaaccaaaaccgtagttgaaggtctaattacagtacataaatattcacgaaaaggcattttaatcttaggtaaaatgatgtacagcctgcctatttgagttaaaatggcaaatacaatcattgaaatgtatacggagggcggagacatgccggtagaaagcgatatataggcacttggtaaagtcaacaacatgatcgccccctccactatttgaaatctttttatattaccagttgcgtgaattcctattataataggattagcaaacgagtttacaagtaaataagccattgtaagcttgaaaaagacagatgtatattcaggtacatcaatcagccacattttcaaaatgtaatcaatgacaaaatatagaggaacagctatcaagattaatatataaaagaaacttatcgagcttagggcgattaatttgtgcataaaagcaagattgttctctgcgtatgattttgttatctgaggattgatagccattcttgcattgttggcaaaagcagcaacgtaatttgacagtgaagtagagagccctctagcagcatttatagctggaccgcaaaagatatttaggagaacgctcattccttgttcaatagtcattacagaaagatttcctaataaattccatccagcaaaagagaacatcttacgaaaaacaggtctgtcaaatcttagtttacacttagcctcttcaaaattccgtctgcaatacaccaagtaaaaaatcctatcaaccacctgtatcattgttaccagaatagcgtagaaaatcaatttatctccgctaaagaaaaccagtactatcgtagcgagcaacttcattgttgaatcaaaaattgagatataagcaaatgatttcatcctttcatatgctattataagagccatgtctggtgtagacatgatattcaggatggtgacaacagaaactgaatgaagtactaaaattgaggctgtaactcggtcataaggtaagtttaacttgttgtaaaccatccattgcccaaatgactcaattaaaaccaatactaaaagaccaataccataatgtattattctcgcttgattatatattgtgcaaagcttttttttattattttcacctatcgcaaaggctatgaatcgtgatgttgcaacgctcatggttgtatttaaaaacacaaacatagaagtaaccccgcccacaacattatatattccataatcaataacaccaagtgcgtccaatacaagtctactggttataagccccattaacattgtaatgaacatccttacgtataacaatagactattttgagcaatacgttttttatttgtttctagagttcccattatagatgtagtttctgtctaaacaatctcaagattttattagaaacgtgatgaagacctattaaatatttaccaacttgagggtaggtatagcaaatacaagcttctgcccataattcatgaatatctccccctaatatatacttgaaagttttagccaatgagatcttcacagacagcccagaggttgtaccctcttcctcactaaaatatttcaagaaggatagtgcactgaacttaaaataataaggtttatgaatcaatgcgtataataatgtttcttccatttctgccaaaacatcagaagcctcattcctcagtcctaattcactaaggcacataataattctattggaaacggttttattagtttctgcaatctcttcattgtctattctactatcaacagcaaataaagggactgtttgtccgttatgcgttttatatccaatagttgacaattgtggatttgctgacaaataatcttcaacaaaagaaataaaatatttaggtaattctacattaggattatgagtataaaagtttaccggaaagtaattacgacatcgtttaaatgtgtgccaatagaaacaaggtgcaggacgaataccgaatttctgttgcaataaattcaatgcgttcaatccacttcctttccaaccgacatccacaaaagcatataaaatatcatcaaacaagccctcttgataaaaatagtcctttatgttctgctgctcattttttatgtgttgctccaatttcctatagccatgcttattgttaattaatgcaaacaactcatctagtttggaagatgcgctattccttatttcgctttcgttatacaaagaataatctacgtattcatttagcttgaaatattcaacaaccttgttaatacttaaattcttgctctctaatccaaaacatcgttcaatatttcttctatttattttatctatagttggaagatacaatgatttccgggaaacatataaatatttcaattttacatctgattcctgactatttagcatttgagccatataatacaaaatccaactatctctcgccaagaagtatagtctttttatcccttctctttttgatacacttaagacatcaagtacgaatgcaagacaaataggcacggctacatctgctgaaaattcaatgatgtcaacatttctattacccattaatgaaatcctcgcaaatcttaaaacgccaataaaactgccatataaataggccatccttttcgccttatacttgtttagtatatattgctctattggtgtatatccggtattctcaattaaataagcatcaattcccttcttttttgcatttatatagtcacttcttttattatcaccataatggcaccaataatcaggcatcaaatcttttcgaaccagctcgaataacataccgtccgatttggatacaccatactcagaagatatatagattctatcatagggctctgaaaccccttctttttcgagaacgcctctaataaaataagaatctaaatacatgtcagatataaaagcaatcacaaaacctttagatcgtttttctttaattgtcgatactatgagttcattcctatacagattctgtctttcaatctttaactcgcattttcttacgtattgactgttgtattctgaaaaacgttctgttggataacagcaataaatatcctctatggttggatgtgatttattcatttccaacctcaccatcttttctgcttcaagtctccagtttaaaaaactttcttgcttcaccacagactcgggataaagtctccttgacaacaaaaagaaaatattgtttggatatccacattttcttattaaacaagtatcaaatatatcgaatgacgctaatttcatctttcagcaattgttatatattgcgttaatgcctaaaatctacatttaaccataatcggcttagtgtattttgcaatcttcgagtatatacccattttggcatacttacgaatattttctctttttattttttgcataccactctgcaaacattctcagccctacccttaatggggtagagggcttgaagttgaaatcacgctctaaaggagtgatgtcagcgtatgttacggatacatcaccaggctgcatagatacaagctctttgtgcgcctcgaaatcatagtcggacggaagtactccggcacgtaccagttcttcctgtaatatggtgacaaagtccaatagattttctggacgattgttaccgatattgtacacacaatagggcgggatgggcagaccatcctctccattactgcattcgggagcatgctgcatcacgcgtatcactccctccactatatcatcaatataggtgaaatcacgcttgcagttgccgtaattgaatatctttatgttctctcctttcactagcttgtccgtgaatccgaagtatgccatatctggacgtccagccggaccatatactgtaaagaagcgcaatcctgtagatgggatgttgtatagtttgctgtatgcgtgcgccatcagttcgttgcccttcttggtggcagcatagaggctgactggattgtccactttgtcgtctgtcgaatagggaaccttctcattgcttccgtacacactactggaacttgcatacaccagatgctctacacctttgttcccgtcatcatacgagtgacggcaaacttccaatatgttgtaaaatcctaatatattgctcttgatatacgaatcaggattagttatgctatatctcacacctgcctgagctgcaaggttcaccaccacttccggttgataatcactaaacagagtttcaacttcttccttgtctgctatatcgactctcataaaaatgaagttatcatattcggaaagtgaatgaagccgttcttctttcagcgacacatcataatagtccgtcatcgagtcaatgcctaccacttttacatcttccacatcctgcaaaagtctcttcgcaagatttgcgcctataaatcctgccacaccggttatcagcacggttttgccgtgcaaagatacgtttttattaaatgccatcttatctatggttgtataatccatacagcgtatatactatttatcaagcaattacgccatatttttatttaaaataaaattcagtttatattttgataagcactcaatttagggcatactcctttaataaggcatttttcattgctctaaaccaagttaagtttttaacattggcttgtgaccaaggctatccgctcaggagccatgttcacgttatggtaagttacggacatctgaagctcccacgccactgccaaaatgcagcgagttgtttcgcttcgattatacattacagcttaatcatatccaaacctttggtaaatactcaattaaaaattagcctgcctcatctattttgtctttgacgtactttctcccctattgagaagttcaattcctctgtcaagttcctcctttctataaaaagcacttaaataatatttcttgtcgtcacccttcctgtctgtcaaaaagaactcaaattcacctgtagaaaatttgtttttcggtaagtctgctttgtctatgcctaattccaataagcaatgcttataatcaaaacttatggagagaagattatggtataggttgttcggcatacaataacgctccatcagactcttcattttgggatgatggaatccattgtaatacaaataacaagctcatatcaagactctttaaattttataaatgttaaattttgtattattcttcctcactaagccaatggttatgctcaataaacttcagaatagtataaagatgcctgaaataatgttcgaatgcccctgaagtataataatcatccaacgcatccacgctttttgccccaagaactccggacggtccatttacagattcactatgaccagtgctatcgtgaacaatatgttcgcaaacttggaaaacataataaaacatgttacgcccattatattcaatggagagtactacctctttgttttgcccgtcagggccgttgggattgtcttctttgacacttattttatttgtttcctttgcatacaaatcattaactatgtactgctgcaactacatcatattaaaaaacttattttcaaaatgttacaaccgtaaagtttcgctctgcaaagcccccttcgcgttggacttcctattctttgcgctacatactctcaattcatagcgttgctaatcgaacgtcacaatcatgcaggcaaaagcaagggcagaaacaagggtattgacagtgccaaaactgtctccgaacaagtccggagcacagttgcaaccatccacggagaacagacatagctgacttaacatccatttggtcagaaagaagccgtttatgcaaatgaaaagacatgctaatattgccatgttgccatggatcaccatttcgttgcattgggttctttctcatctgcgcgtattttttatatcaaaatattatcttaacaatgaacatgcgtttattatctagttttgccaatatttcgccaccaataatgccacgtttacatgttggctaaatggaaagtaaggctgtaaccctcagtccctcctaaaaatatccctcgtataaaccttttcctccacatcatccagacaagcgtcataacggttggcaatgatggcatggctctggcctttgaaggcatccaaatcattcaccactttactaccgaagaacgtgctaccatcttcgagtgtcggttcatagatgacgatctccgcacccttggctttgatgcgcttcattacgccttggatgctcgactggcggaagttgtccgagtttgatttcatcgtcaggcggtacacgccgatgacacacttccgctcctcggaaggattgaagtctccacggttgtagtagtcgtagtaaccggccgtgtgcagtacgcggtcggcaatgaaatccttgcgcgtgcggttgctttccacgatggcctcgataaggttttcaggaacgtcttgatagttggccaacagttgcttggtgtctttgggcaggcaatagccgccgtaaccgaacgaagggttgttgtaatggcttcc

SEQ ID NO: 2 - DNA, 12131 bp, unidentified
  source 1..12131: mol_type = unassigned DNA
tcagccattgtaaacctccaaaattctctgtttaaatttttctctgcctactctgtcgattgtaaacttaaagcgttcgctcggctttgcgttttctgcaaagaagttaagagcggcatcacagatttccataagtttttctttattttcgataaacggaataattgtttcgcccttgttgattgagttgccaaaaagtccgccgaatgaaacaatgtagccgtgaattgtgtcccacgcatctgtcgggcaagccttgaagcatctgccgcagtagttacactttgaattgtcaacggaaattttcccgtcctcaagagtgattgcaccctgacggcaggcctttacgcagactccgcagccgatacaagcgtcctctttccatgaaaccttgattccgcccttgataccgacatcgttttcctcagctttaaggcagttgttctgacagcctgttatgccgaacttgaacttgtgcggaagttcctttgcaaaatatctgtcgtcaagctcctttgcaagagcgtatgtatcaatacaaccgccgggacagattgcctcaccctgacaggctgtgattgttcttactctcggaccgcaaacacccggctcaacatcgccctcggcaagtgcgtccttaactgcctcaacatcatcaagctttacaaaaggaatttcaacactttgtcttgatgtgaggtgaacataaccgtcaccgtagttttccgaaacctcggcaattttcgcaagctgtttagctgtgaggttgccgcctacaactctgagcctgagcgaaaaattatttttctgtttttgacgcataaagccgccctttttaagcgtagcataatcaactgccatttctcttactcctccgattcattcacatttatactgtctacagcctgtttgaagtcagactttaaatcctcaatatcctcaatgccgacgctgattcttattgtgtcatcaaacacgcctgcgtttcttttttgttcttctgttccgtgagcgtaaattgtgcttgacggatgaattacaagtgttcttgtgtcgcctatatttgttgcatttgttgcatacttcaaattgttgataagcttaaacgctctttctttacttcctgccctgattgtgaggattgcaccgccttttcccttcaacagcttttgacaaaggctgtaataaggagaagctttaagagccggatagttgacttcgattccgtcctgtgtttcaaagaactttgcaagctcaagagcgttgtaacaaagacgctccatacgaagtccgagtgtttcaagtccgacagagttcataaaagcattaaacggtgcaaggcaacagcctatgtttcgccagataccgtttctcagctttgcaacatatgcaagccgaccgaattttttgtactctgcaagacctttgtatctttccgtattccattcaaaattgccgctgtcaacgattattccgctgattgaatttccgccgccgttaatgtactttgacgaggaatgaacaacgatgtccgcaccgtgttcaaacggccgaataagatacggagttgccgttgtgctgtcgataatgagcggaacaccgtgtgaatgaacaagctctgatacagcgtcaagatcgacaatttcaagtccgggattgccgataagctcggcaaagactgcttttgttttttcgtttatgagcggttcaatctcatcggctgtcaccttgttgacatatcgtgtaacaattccgaaaggctcaagatctccaaacaaatcaattgttccgccaaaaagacctgcgcttgcgattacttcgtcacctgattcaagaatgttcaggagtgccatcgtaacggctgccatacctgacgaacacgcaaccgcaccgacaccgttttcaatggaagcaattctgttctcaaaagatgttaccgtgggattgcctattcgagagtaggcgtagccgggagctctgttgttgaacaccttttcaagctgttcggcagagctgtgcgaaaatgcactgacctgataaatgggagttaaggttgagccaaaagccttttcaccgttaaaccccttgtgaagtaatgcggtgttaaactccataatatcaccaaccccttaatttcatacaaccaacccttttgtgtaagaccgtcgtaggaatttgtctgacggcttcgattgatataggtagattatactccattaatcatagtatttcaataggtttaataagattttacctcttgcacaatttttctttaaaatttgccgtaattccattatttcctacaatgcatatttgtttagtaggtattgatttttaaattaaaaactgttaccattattatatactctttcagcaaacaaaacgcacggaaaggagatgctaaaattgaagatttcaacaaaggtggaattcggaatcatcgctgttattgacattgcgtcacactcactaaacggcaatgcggtaacaactcccgaaattgccaaacggcaaaatatatctaaaaagtatcttgaacagattctctcatcactcagctcggcaggacttgtgcgaggtcagaagggttcaaacggcggttatgttattacaaaaagccttaaagagattacattcaaggacatcataaacgcacttgacgcaaatgttctgagcaatgtttattttaacactcggggcgatgatgatgttatgataagacttatcgacaacagtctttggtcaaagatgtcgtcatatcttcaggaatatgccgaaagcattacacttgcacagctttgcgatgagtatatcaaaaacaatgaaattgaacacaacattccgatgtactacatctgatagattacaaacaaaaagccaccgatttttatgtcggtggcttttcttgtttttatcggattttatttaaagctttcaataacctgacaggttttttcaatgtcatcttccgtgtgcgcaaatgaaacaaacatcgcctcaaactgtgacggtgcggcatagattccgttttcaagcagatgattgtaatactcggcatatctctttgtgtcggctgtggttgccgtgtcatagtcaacaacctttttgtcggtaaaaaatgccgtcataatcgaacccacacggttcacattaagtcctgcgttcttcattgcattttcaatctttgcagacttgcgttcaagttcgggatatatcgaatctttatttttttcaagaattttaagcgttgcaatgcctgcacttaccgctaccggatttcccgaaagtgtgcctgcctgataaacagaaccgagaggagcaacacaccccataatctcttttcgacctccgtaaaccgcaagcggcattccgccgccgacaatttttccgagggttgtaaggtcgggttttactccgtaaagcttttgagcaccgccgagtgaaagtctgaagcctgtgattacttcatcgaaaataagaagtgaatcatgcttttcagtgatttcacgcaagaattttaaaaatccttttttgggcggaacaacacccatatttgcggcacaaggctcaacgattacacaggcaatttcatcaccacagttatcaaaaagctgttgcacggattcttcatcgttatattttgcaagcaaagttgtgtctgtgtagcctttcggcacacctgcgctgttcggaattgaatttgtcagaagtcccgaaccgccctttacaagcaagccgtcggaatggccgtggtaacagccctcgaattttacaattttatccctttttgtaaagcctcttgcggcacggattgcgctcataaccgcctctgtgcctgaatttacaagtcggagcatttcaattgacggcatattttcgcggataagctcggcaagttcaatttctccgctgtggcacgcaccgaatgtaagtccgttctgacaggcagaagtcacggccttgataacatcgggctgtttgtgaccgaggatattcggtccccatgagcagatatagtcaatgaactcgtttccgtcaacatcataaatttttgaaccctctgcacggtcgataaaaagcggagttcttccgaccgaacggcacgcacggacaggactgttcacaccgccgggcataaagtcaagcgcctgtgcgtatagcttttcgctttgagttgtattcataaaagcacttccctatttgtaaaattcgtcaatccattttgcaacatcaagtgcgtggtatgtgattatgatatccgcacccgccctcttgattgcaatcatattttcgcacacaattttcttttcgtcaatccagcctttttcggcagccgccttgaccattgcatattcaccgcttacattataggcaacaagcggaaggtcaaagcgttcgcttgcggcttttacgatatcgagatatgcaagtgcaggcttgaccataaccatatccgcgccctcgtcaatatcatctgcaatttcacgcagagcctctttgccgtttgcgtaatccatctgatagctttttctgtcgccaaattcgggggcagattctgcggcatcacggaacggtgaatagtagcctgatgcaaattttgcactgtatgacataatcggagtgttgataagtccgttttcgtcaagtgcgtttctgattgcagacactctgccgtccatcatatccgaaggagcgatgatatccgcacctgcttttgcaaggctgacagccattttagagaggagcggaagtgtttcgtcattgagaatttcgtgaccgcaaacaacaccgcaatgaccgtgacttgtgtattcacaaagacagacatcggcaataataaggagtgacggataatttttctttatgtatctgattgcgttctgagttacaccgttatcgtcataggctgatgttgcaagctcgtctttatgctttggaattccgaaaataagaagtcctgcaatgccgctgttttcaatttctttaagaatctcgtctgatctgtcaagcgaatattggtagacattcggcattgaatcaacaggctttttaatattttcaccctctgcaataaaaatcgggtaaatcaaatcgcccttgtcaattcttgtttcacgaacaagactgcgaatctgctcgcctgctcttaatcttctgaatcttctcatatatttatccttctttcaaagttacttccttgattatagtttccaaaattccgtctgtgtccttcgtttttgcaatcctgaacgaatttacgccgtgactttcaagtgcctcggcggtaatttcgcctatgcagacaacggaagttctgtcggaaataattgctccgccgtcaaaaaatgtgttcacacccgacgcactcgcaaaagtgataaaatcggtatcaacaacggaattatcctgttttttctcaagctgaatgtcataggttttgacatcgtcaaagtcaatgttgctatcagccaaaatccgagtgagttcgggcgaaccgttttcggcacgcagaatcaatactttttcgccgcttttcaccgtttttgcaagcaaaattccaagttcacgggttgtgtagacattcggaatacagtcggcaaaaattccgtacttttcaagtacagccgcagttcccgaaccgatcaccgcaaacttaatatttgcaaaactgcggatatcctttttcagcttacgcagtcggctcataaaaatttctgcaccgttcatacttgtaagcaccacataatcgtagccgtcaataccgtttattgcattgtcaaaaacttcgttgtcatgatattcaatcacctttaaaaggtcgtgccttttgacctctgcacccgaaagcgtgagcattttgccgagcttgtcggagagctttctcgtacccgtaactgtaaccgaaacatttttcaaaggtcttgtaattgtgggggtaaagtcaaattttgcggtttcgccaaccactataacggcaggcggtacaacctctgctgatttaacattttcgcagattgtcgagagagttcccctcacggtctgattttttgcacaagcgccgtttgacacaactgcaacgggagtatcctccgatttaccgttacgaatgagattttccacaatgtcggggaggtttctcaatcccataagaaagacaagcgttccgtcggtttcggcacacttctttatattttcgggcagggtgtcctgtgccgtatgtcccgtgattatatgaacacttcggctcagatttctgtgtgtaacagggattcccgcaagttcgggaacggcaatcgaggaagaaattcctggtacaatatcaaacggaatatcggcacttttgagggtttcaatctcctctccgcctctgccaaacacgaacggatcgccgcctttaagacggacaaccgttttgccctctctcgccttttcaacaaggatttcgttaatattttcctgcttttcgctgtgtctgcccgaacgctttccaacacagattttttcagccgtttcgggtgcaaaaccgagcaatgactcatctataaggctgtcgtaaacaagcacatcgcaatgtctgatatactccgcaccacgcattgttatcaagtcaaaatcgccgcatcccgcaccgacaatatacactcttccgttactcaatttcgctcaccagcctttttgcaagtttgattctgtttgaaatttcatctgttccgctgactgtttttttgcagtcggcagacagagaaagcattatcctgtcgttttcaacctttgaaattgcaccgacgggagttgaacaatcggcattgaggctgagcagaacctgtctttcggtttcaaacgaaagcattgtatttttgtcactgatttcagcgagcattttggaaacctcgctgttttttctgctttcaatcgcaactattccctgacacggtgcagggagaaattcatcggtatcaaacggagttaccgtgtaatcctcaccgttgcaaaggtcaagtctttcaagtcctgcctttgcaagcacaagagcatcgtactcaccgttcaacagctttttaagtcgggtgtcaacattgccccttatatcattgaattttgcattaggatagattttctctgcaaaaagctttcgccttaaactgcctgttccgatgacaaagccgtccttaatttcaaaattgttgcgtgtaacaaaaacatcgccatagctgccccttggcaacacggcggaaatttcaagtccgtctgcaatttttgtcggcaagtcctttgcactgtgaacggcaacatctgtttctccgctcaaaagtgacatttcaagctccttgatgaacacaccttttccgcctaaagctgtaagcggtttgtcaagggttttgtcgcctgttgtcgagatgtaaaccgtttcaattttaacattcggaaaacggtttttcagcgtttttattaacatttcggtttgtgcaagggcaagcctgctttttcttgttccgatttttatcgtcattgttcgtattcctcaatcaaattttcaacagttttttcatcgggttcaagtcccgaaaggcaaagcgaaagaagctgttcaaaaatcttccttctgtcatcttccctttcaaccttctccttaattatcgggcgatatttccacaaaacctcggttgtattttcgttcatactttcaaggattcccacaaaaagctctttaagatattttgcgtaaatcggacttttgcccgatgttgtaattcctgccgttatgccgtttttgcttgcaattgcaggaaaaataaatccgcatttttccttatcgtcaactgtgttgacaagaatatttttctcattgcaaagctgaaaaattctgccgttgagcgtttcatcatcggttgcgctgacgacgcaaaacgcaccgtcaagatcgctgtcacaaaatctgcggtcaatgattttcacatgcaaagccttgatttcatcgcagattttcggggcaacaacggttatgtccggcgaaaacggcatcagcttttcaatctttctgagtgcaaccgtaccgccgccgactacgagtatttttttgttttcaatgtcaatataaaacgggaagtatgccattagttattctctccgcactttctgtagatttcaagaattcttgcaaattcttctgccgaaacatcggatttcagcttataaatgaatttttccgaaaaatctgacggcattgcgtttttcacagactgcatacacggcagaaattcatgaaaggcaaggtcttttttgagaacttcaatttcctcaaaaattatttcttttgcactttcaacgctgtcaagtttgagagcattgttctcgtttgcaagctgttttatgcagtcaatgttaatgagccttacaccgcttattttgggaatattttcgtcaatgtcgggcggtacggcaaggtcaataaacagccttgatttttcggtttttatattgttttttaaatcataatatgtaaccgtgtaatgcggtccgcttgtggcactgattacgcagtctgcaaaatcaaacgctttgtatctttcgtcataatcaatcacattcgcacccgtattttcaaaaacagtttctgctttatgttttcgtgaagtcacggtaacgcttatacctttgtgtgacaaaagatttttcacaacggttgagccgaccttgcctgttgcaccgattacaaggacattcagctcaccgtctttgaattttgccgcctcgtttgacgcaagagttgcaacggaaaccgatgtttttgaaatggcggtttcggtcttgattttttttgcacaggcaacagccgcctgaacgcacatattaagctcataggcaaccgtgccgttgtcctttgcaaaggcataggctcgctttagctgaccgagtatttcatcttcaccgataaccattgattcaattccgcctgccaccctgaaaagatggagcaaagccctgtcaccgtagaaaagacagatgtatttttttagttcgtcaaaatcaattcccgaaaaatccgaaagaacggtttcaaccgttttcacagatgattcgtcaccgcaaaaataaacctcggttctgttgcaggtgcagagaatcaaacactcgctaatattttcagaataaaaaagttcgtcaagaaatgtctttttttgttcatcgggaaaggcaagcttctttctcacaaccacatttgaagttttgtgacttacgctcaggcagtacataaaaatttcgctccgttacaaataaaatccgacagtaaaatacacttttaaagttattttattactgccgacagctattgtcaattaaagttagtcagttattttaccactaaacctatctgtttggtatgttttgattatactacattcaccgttccatgtcaataaatttcgggttttgcttgacaaaaacagtgattacaatataaactatacaaagatacctcccgtcagcaaacagcgggagttttattccgatgtaaatacttatggtaccgataggaatactaaatgatttgaatatgtattaaggagcgtttttatgcttaatcagttttcaagaacacagcttttgattggcaaagaagctatggacaggctttcaaaaacaagagttgcaattttcggaatcggcggtgtaggcggatacacggcggaggctcttgcgaggagcggagtcggcgagcttgaccttattgacgatgacaaggtgtgccttacaaacatcaatcgccagatttttgcaacaagaaaaacagtgggcaaatacaaggttgatgttgcagaggaaaggctcaaggacatcaatcccgaaatagtcatcaacaaacaccagaccttctacactcccgaaacctcggaaaaattcgacttttcaaaatacgactatgtagttgacgcaattgacacggtaacaggcaaaattgagcttgttatgcaagccgacaaagccaagacgcctatcatctgttcaatgggagcaggcaacaagcttgatcctacagcgtttgaggttgccgacatttacaaaacctcggtctgtcctcttgcaagggttatgagacacgaactcaaaaagagaggtatcaaaaagctgaaggttgtttattcaaaggaaaagcctatcacacccgttgacgatatggcaataagctgtaaaacaaattgcatatgccctccagaaactgagagaaaatgcacccaacgcagacaggttcccggcagtaccgcatttgttccgtcagttgcaggtcttatcatggccggcgaagttatcaaggacttgattaagtaaactaatatagttgtcttttcattttaatttgctattgttaaaaattgcaatttatagtataattatcctaataactatttagaggttgcaaaatgaacagatatataattattgcaggagttaacggaacaggaaaatcaagtttgcgtggtgttcttgaaggacaggaagttttgcttggtcatataattgatgctgatgctattgcaaaaatgaacaattacgataacataaaggcgggtaaaaaagcagttgaagaaataaaatactgccttgataacaatatttctttcacacaggaaaccactctttcaggacacagaaccgagaaaacaattcagcaggcgcgaaaacagggctactatgttattatgtactatgtaggtcttaactcaaaagaagaaagtcttgcaagaattgaaaacagggtacggaaaggcggtcatgatattccgaaaaatgatgtgtgcagaagatttgataaccgcttcgattctttaaaaaaaattgttccgctgtgtgatgaagtaattttttatgataatgaaaacggatttattaaagttgccgaaataaaaaacaacaaatttaattattcaaacggttacagacctaaatggattacagaatttgaaaataaactgaaattaggctaaggaaaagcaaagcaccgagaaaaattgccctcggtgctttgcttgtttttataggattttttacagttgatatcgtgattaaacagattagatgataaccgattcttcctgaagtgatttgttacggagaatctgaacctcatcgcctttggtaataaaaagccgataaagaaatacattgaccttttccccgacggtaatcggagtttcgtcaagtcctctgtacgtataaaccgtgcctccgtcacattttacggcaatttcgatattgtcgcctctgaacgaagtctttataacctcaccgacggatacagacgctttttgtgcggttgtttcgctgagctttgtcacctttacaaattcgggacggataactgcacggtcaacattatcaacaacatcaaaggtcttgaactttgtataatcgtcaagcgtagtagtctgaccgaaaaatgatgcggaaaatgcggtttggggatgctgataaatgtcaatcggcgagcctatctgctcaattctgcccttgtttgtaattataatttcatcggcaacctcaatagcctcgctctgatcgtgggttacaaatatacttgtaacgccgagcctttcaatcatatccttaagccatgagcgaagctcctgcctaacctttgcgtcaattgcggcaaagggttcgtcaagcagaagaagattcgggtttggcgcaagcgctcttgcaaaggcaacacgctgtttctgaccgcccgaaagctggcttggataacgcttttcaagtccttcaagaccgataagctttacaagctcgtttactctttccttgataaatttcttgctctttttctgaattttcagaccgaaagcaatgttatcaaaaactgtcatatatctgaaaagagcgtagttttggaatacaaagcctatgcctcttttgcttgcgggaatatcgttgacaacctttccgtcaattacaatttcgccgctgtcgggattttcaagtcctgcaatcatacgaagaattgttgttttgccgctgcccgaaggaccgagaagtccgataagcttacccttttcaataccaaagtttacattgtcggatgccttgtaatcaccgaaagttttatttatatttttaagctcaacatacat

SEQ ID NO: 3 - DNA, 15883 bp, unidentified
  source 1..15883: mol_type = unassigned DNA
gtgtttatgatgcagtcagaacgtttcagtgaagaagtcaaaagtttattggaagccagtcagcagcaagctgtcatgaactataaccaagaggtgggaaccgtccatctcctgtcagccatgatgagcgaacctgatagtttcctccattatgtcttaaagagccttcatatcgaggaaaacgacttcagtcgtgaccttgaccatttggtgaaggccgtcccgtctgtgaagggaacggaccagcttagtatgtccacggggatggcaagggtctttgccctcgcggaaaaagcagccggccaaaataagattaccggagcccatctgctggctgccctctgtgaagatggcgatagcgatgtggtttccctctgccgcaagtacggcattacccgttcggccatccgtggtctcgtcgaccgctatgaaggcggcaaggacagtgacgaaaaccgcaagaccttggaaaaatatgggcaggatctgacggctaaggcccgcaagggagcccttgatccggtcattggtcgtgacgatgagatccgccgtaccgttgagatcctgtctcgtcggaaaaagaacaaccctgtcctcattggggaaccgggtgttggcaagacggctattgtagaaggccttgcccgccggatcgtggcaggcgacgtgcctgaatccttaaagaacaagaccctctatgccctggatcttgcgtcccttgttgctggggccaagtaccgcggtgaatttgaagaacgcttgaagaaagtcctcaagattgttgccgattcggctggacagatcatcatgttcatcgatgaactccataccgtcgttggagccggtgcctccgaagggtccatggatgccggaaatatcctgaaaccgatgctggcccgcggtgaactccgctgtattggggccacgacccttaatgagtatcgaaagtatattgaaaaagacagtgccttggaacgccggttccagcctgtccttgtcaaggaaccaagtgtcgaggatacggtttccatcctgcgtggcttgaaggaacggtacgaagtccatcatggggtccgcatcaaggattctgcccttgtagctgcggctgcgctttcaaaccgctatatcagtgaacgcttcctgccggataaggctatcgacctcgtcgatgaagcggcagcccgtctgcgtacggaaattgattccatgccgacggaactggatgaaagccgccgccgtatgcttcagctccagattgaggaacaggccctttccaaagaagaagataagcagtcggctgaccgtctcgaaaagatccgtgaggaactgaaagagatgaaagcaaagaacgataagatgacagctcgctggcaggaagaaaaggctggcattgccaaggttcgccaggtcaaaaaagccattgaccaggtgaagaaggacatggaaacggccgagcacgactatgatctcaacaagcttgccgaactcaagtatggccgtctgccggaactgcagaaacagctccaggaactggaaaaagaagagcatgcagaagaccgtcttctcaaggaagaagtcgacgaagacgatatcgccaaagtcgtcagcacctggacggggatccctgtgagccgccttatggaaggcgaaaagaagaaactggctcacctggatgaaatcctccatcagcgggtcatcggtcaggatgaagctgtgaaggctgtctccgaagccgttatccgcgctcgtgctggtatcaaggatcccaataagcccattggttcctttatcttcctgggcccaacgggcgtcggcaagacggaacttgccaagagccttgccgaagtgctcttcaacgatgaaaagagcatcatccgcatcgatatgagtgaatatatggaaaagcataccgtaagccgcttgattggtgcgcctccgggatatgtaggctatgaagaagggggtcagctcacggaagccgtgcgccgtcatccttattcggttatcctttttgatgaaatcgaaaaagctcatgccgacgtcttcaacgtgctcctccaggtccttgatgatggccgcctgactgatggccagggccgtgtcatcgacttcaagaataccctcatcatcatgacgagcaaccttggatcccatgaaatcatggaacataaaggtgaaatcagccatgaagaagtgcgcagtatgctgatgaagttcttccgtccggaattcctgaaccgtgtggatgacattgttgtcttcaaaccgctcggcgaggatcagatccgcagcattgtccgcctcattctgggtcagctgggcaaacgcctgcaaagtcagatggaactcacccttgaggccactgatgaagctgtcgatgcccttgccaaggaaggcttcgatcctgcctttggcgctcgtccgttaaagcgcctcatcgttcatagtgtggaaaccatcgtcagccagaagattgtccaaggagacattgtaagcggcgatcatctgaaagtcgtcttgaaaaacggtaaaattgacgtggtaaaggcgtaagccttgactgctgggaaggaccccttcttcccagcatttttacgtttattgggattttttgggaaaaacctttgtgaactcgtcgtaaatgagttcacagagtaggggttccgttgtataataagaaggaatttgatgaatcgccaaggaggatttccatgaaaaaattactcacccttgtcacgcttcttatgacgctccttgccgttccttttacggcaagtgctgagcgcagcggcgccaaagcaagcggcgttcctttcaataagaccaaggttgtctatctaaaaagtctcgaaaagactgatggggaatttgttaagaataacccccaattcagacgcgatgaggatgcggtccgcctcacctatcttgggctccagaaatccttgaaggatgaaggggtgacgctttataacaatccagtcatgctgcccgaccgttacctgagaaagaccctgaccatgcaggtcgttgtcaattttgcagggactgtgcccctcgaaggagaagcggctcaggaagccatcaaggacaaacgggaaccggtggaatcccttgtctccctttccttccttgtcaccaaaggagaggcaccggtctaccgtatgaccgacgtgcgtacgagtaaggaaaagaacttggaaaaactcatccgggatatgacaaaagatgtagcaaatgaaatcacgaataatcgtatgaaactaaaagatgtaaaataagaacgccgagaggctgtgagaaatgagtacatcatttctcacagcctctttttgtatggcttgggagtgggggattatgatgttcaggtggtgtgatgctgtgatgcagggccgcactgctgttctgccactaaaatcggtgcagccgaatcttttgtctttggaatttgaatccatcaaaagactgtacagccgaccctaaaccgttccttctttaaagggggagggaccgaatccattcgcccggaaacatggccctgcgcccttgtgctttgccccaatgcgggcttgggactcgggcatatttgccttgctagctgcttgcaaggaaacgtcgctgcggtttctcatccaccaccgaaatcgggaaatccgaattggataaccgtatggatatttcccacacaacatgagtgctaataaattatattatcggccccgtcgtacacgaaaacggttcctttacctccgcagctgcaatggatttatactggtgcggctgtcaaaggcggggtatcgttttcgagtcaatgcggaagatggctatagaggatcagccgtatcatgttccgtctttttgactacgcagatgggccgatatgggcttcaggcgaatcatgtttcctttaaaaaccgaaaaaattccaagaagtggcccatccgccgaaggcatttttttgggcgtcttaaaaatgagattgtggtacgtaccttcttgcgacggggcatgtcatcatcggcatcagatcaatctttttaaagccaatgacgacacgtagccgcatcgaatcggcgcgatttccgcgccgagtaaaactacagacctctacccttgcgacaaacccatttaaaagatataattctatgagtatcatcgtgagaccagcccggttatccgccctgtgtcccccttgaggaaaggatggacgatatctatgccgatacctcttgctctttgggctgctgccggaatcggagcagcgggcatgaagctctacatgaacctgaacagtcctgaaaccgtcgtgcgcaatgcaacggagcgttataacgaagaacgctataaattctataaacaaatggaaaggctcgttcccctcatcgaggaattgggccgtttgaagttatctacgtggtccaagtataccgcgatgtttgacacgctggataagctcatcaacctccccggtcattatacctataagagccacaagaacctccacatgctgccccaggatgtgcgcaagctgcggaaggtggcagacgtcgtaaaacgaatccatacggatcatcttgatattgaagggacagggatgctgactattgttgcccttcaaggcggggcggccagtgattacagccagacggccctggccgaaggggagtcgcgcctcattctcgaacaaatcatggagcagcccctttatacgtcagaatgcggcgtccttgacgaagagcaggtcctgtgcgccgtcatggattttcccaaagtcatgccccgtggttatttcaaggaccgtcatggcagaaagcgggaacgcaaggtttccaaacgggcggcccgccgctacaaggaattgacggataaggaatcggtccgtcttgctgacgcgacggcgcgggctgatcgcctttttcatgttgtgacccatgtcctgggctatatgaagccgctcaaaaaggaacatgatgaacagcttgatttttttacggccctcatcaagaaaaacaacgattacgatcagtttacgctcgaacagaaggatcgtctcaattttggcgttaccttaggatttgtcctgcgtgaactggcccggaccgatatcatcatcaagaacgggaatgttcccatcatcaatacttcggccctgcgggcggtttatgcccgggccaaagatttgatgcctgcggaagaaatgccctatcagatataagaaaggaagaattctatgatcagtacaaaaggtttcaaagcctatgatatccgcggcgtcgttcccagcgatgtcaatgaggaacttgcttacgccttgggccgcagtcttgccttttacctcaaggcaaagacccttgtcataggtcacgacatccgtctgagcgggcccagcctgcgcgacgccgctgtacgtgggatgcgtgacatggggacacatgtcatcgatcttggtcagtgcggtacggaaatgatttattttgcggttgcccatcttaaggctgatggcggcatgatgattacagcaagccataatcctgccgaatataacgggatgaagcttgtccgcagcgagtcccggcccatttcgggagacacgggattaaaggaccttgcggccatggtcacagatcctgattttgaaacaaaatatcccaaagccgcacggcagggaagctatgagcaggtggacattgtcccaagttatgtggatcatctccttagctatatcgacactaaagagattgctcccctaaacatcgtggctaaccccggaaatggcggagccggtcccattgtaaaggaactggcaaaacacctgccctgtcactttacgtatcttaatgacacgccggacggacatttccccaatggagttcctaatccgctccttgttgaaaaccgtgatgccaccagcgacgctgtccgccgggaaggggccgatcttggcattgcctgggacggggattttgaccgctgcttcttctttgatgaaaatggacgcttcatcgaagggtattatcttgtcggtctttttgcctcttacttcctcaagaagcatcccggtgccaagatcctctttgacccgcgcttgatctggaatacagaggacatcattgcccgtgaagggggctatcctgtccgctgcaagagcggtcatgccttcatcaaggaatgtatgcgtcgggaacatgttctttatggcggagaaatgagtgcccatcactatttcgccgatttttcctactgcgacagcgggatgattccttggcttcttgtcatttctctcatgtcgacatcaggccttagtcttgctgaaatggtggacgctcgcatccgcgaatatccctgcagcggagaaatcaaccgcaaagtggggaatgccgatgccatcatggaatccctgtcgacgcgctttaaatccggcatccaggaccgtatggacgggcttagcgtcgtctatgatgaatggcgcttcaaccttcgtaaatccaatacggaacctgtggtgcgtctcaatgttgaaacaagaggagatcgggaacttctcgaacaaaagacccgtgaacttctggaccttatccagggcgagaaagcataaggaggcagcagtatgaacgttcttgttacgggcggggccggttacatcggttcccatgtcattgatgatctcatcaaatctggttatacaccgattgtttatgataatttcagtaccagtcatgccgaagcggttcccgaaacggtacagcttatccaaggggaccttcacgatttcaccttcttgaagcatatcatgggccaatatgaaatcgatgccgtcctgcactttgctgcaagttcccaggtcggcgagtccatggtcgatccgggaaaatattactataacaatgtggctggtactttgggtcttcttgacgccatgcgggaaagcggcgtcgagtatatcgtcttttcgtcgacggcggccgtttacggggaaccggaccaggttcctattacggaagacatgccccttcatccaacgaattcctatggccggacaaaactcatgattgaaaacatgctgcgtgactacagcatggcctacggccttcattatgtagccctgcgctacttcaatgctgctggcgcgtcccttttgggaaatatcggggaagaccataatccggaaacccatctcattcccctgaccatccaagccgctctgggtaagcgcgatgctatttccatctttggtacggactacgacacccctgacggcacgtgcctcagggactacatccatgtgaaggacctggcctccgctcatatccttgccctgaaccatcttgtcaagggcggtgcaagtcgggtctataatctgggaaccaagacgggactttctgtccgggaaatcatcaatgccgtcaaggaagtaacgggccgtgacttcatggtaaaggaagaaaaacgccgtgctggcgatcctgcgcggctcattgcctccagcgaaaaaattgcaaaggaactccattggaagcccgaacacagcagcgtcaaggaaattgtggagacggcatggcgctggcatagcggccaccccaatggatatcgctgagccttatggatatcctggtaaccgtcgatcatctcgccaaaagctttggcgctcatgatatctttgacaaggtgagctttaccattcgccaaggggaaaagctaggccttgtcggtgtcaacggcagcggcaagacgacgctcctgcgctgcctcatggatccctcctttgccgataagggaaccgtccaatatgcaggggaaattcgcgttggctatgtggaacaggggtttgacaatatcgaagacgaaaccatctgggagttcatgctccgttcctgcccggatattctttccctccgggaaacgatgaaggaacttgagcgggaaagcggcgaaatgacaggtgatgccctaaaggatgtcctggaccgttatggtcgggtggaaacccgctatgcccacttggacggttatcactacgaaaataacatcaagcgggtcctgatgggactgggctatccagaatccacctgggagcacaacgccctggccctttcgggcggacagaaaacgcgccttcagctggcggcggctcttgtcaattcgccggatctgctcatcctcgacgaaccgacgaaccacctggatattgtcatgagtgaatggctggaaaaatatcttcgtgagttccgcggtggtgtcctcatcatctctcatgatcgggccttccttgatgccgttgtggaaggcatccttgaaatggaaggggggaccctgcaccgctataaaggaaactattcccgctatgtggagcaaaaagagctccaggttctctcccagacccgggcttatgaagcgcagcaggaaaccatccgacgcacggaagagtatatccggcgctacaaggccggcatcaaaagcaagatggctcggggacgtcagtcccagctggaccggatggaacgcattgacgcacccgtaaaggaagaatcttttacgctgcgtctgccccatgccgctgaatccgcggaaaaggtcatcatgttggaacacctttccgttggctatgaaggaaacgcccttttaagcgacattgacctggtcctgcgccgcggggaaaaggtggccatcatcgggcccaatgggagcggcaagtcaaccctcctaaaaaccatccttcaggaaatcccgccccttcacggcgaagcaaagattggaaaccgcgtgaagattggctacttttcccaaagctatgagcgccttgattcccagcagaccattcttgacaacttccttacggagtatggcatgacggacgaacagacgcgccgtctattgggcagtatgctctttcgcggggaagacgtctataaaaccatcgggagcctttccggcggtcagaaagctcgtctggtcctcttgaaactggttcttgacggggccaattgcctcgttcttgacgaaccgacgaaccatcttgatatcatggccaaagaagccgtcgaagccgcccttgagacctttgacgggacggttcttctcgtcactcacgaccgctatctcgttaatgaagtggcaggccgcatctgggctgttgaaaaggggcacctcacgaattataacggcaattacgacttctatctggaagaacgggacaaacgccaacagcgggaagcgccgcttccggaatcctctcatcgagcgccgaaggaagaaccggtaaaacctgatggatcaaagcaccgcccccatgaggaaaaatccaaaaaccagcggatctatacgccggctgaggcggaaaaactcctgcctgaagtagaactgaaaatccgtgaacacgaggctctgcaaaaggtcctaagtcagcagattgccgatcctgaaaaccagatggaccttgaaaaaagtgcggcccttgccgcggaatacgaaaaggagcaacagctcattgatggcttcatggaaaaatgggaagctcttatggaggcccttgaatgacagcattatccattgacagtttcataaaagccctgcagggctatcacagcgatacggccttcaacccatgggccgattacgacccccagtgtgacattgggccccaggcacccctcatccgggcagccaatctgaagcgctatttgacgctccgccaaaaggcacgctttctttttattgccgaaggactgggttatcaaggcgggcacttttcaggcatggccatgacgagtgagcgcatccttttgggtcatcaccctaacgtgcggccccatgtggttcttggcgaggattacccttgccaaaggacaagcaatcccgagtcgcccctgttaaaccgcatgcagcgggaaaaaggcttcaatgaaccgacggcgaccgtcatgtggggggaacttgcggcccaagaactggcccctttccagacgattttgtggaatatttatcccttccatccctataaaccatcgggcatcctgacaaaccgcacgccgacgccaaaagagcttgacgaaggcattgactttgctcgtatgatccttgcccttgtgccggggatgcgcgtcattgccattggccgaaaaagtgcggacacgctccagaagtatggcgtagcctgcgacgcggttcctcatccttccatgggtggggccacgcgctttcgcagcgctgtggacgccttgtttgaatctgaataggtatttttggaggtaacatggaaagcaccattgccagtatgactaaggaagaggtcctcaagatttccctcacgtgcgggaaactgctcctgacaagcggcgccgaaacctatcgtgtggaagatacgatgacgcgcatctgcgccctcagggatatgcagattgctgccgtcagcacgccgtcatggatcatggtgggcgacgaaaccgtggacgggaccagtatggtctcccgcgttacgcggcgggggacggatctcaccctcatcagcgaagtgaacaacatttcctatcatatgcccacgtgggaccattcctttgccgaaaccatggccatgcttgatgaattgagtgcccgcccgtcaccgaccgcactctacagcatggtttggtccggttttgccggcggcttttttgccggcgtcattggtggcaatttctgggattttgtggcagccttcttgggctccttcctttctatggccttcatctgcctcgtgcggcccttccgccccagcagtttttgggaaacgactatggcaggcgtcatcatctgttttgtcacctatatgtgtcatctgttcattcctggtgtcactatggaatttgccattggcggcggaatcatgccctatttgcccggtatggcttttaccaacggaatccgggacttcatggccggtgacctcatgagcggaacgagccgggccggcgaggccattttccttgttggcggcattgccatcggcattgcggccttccttggcatcgtctatctttaaggaggactgccatgaacgtcatcctgaccatcgtcatcaagaccatctgctcctttttcggaaccgtggcttttggcaagatcttcaactgtcctaaacactgcctctataaagcgggctttgtcggcgctgtgggttttggcgtctacatcattctcctcagcggttttggcgtcagttccatgctcagcaattttgctggaaccgtagccctttctatctgcagtgagctctttgcccgctggtacaaggaacctgtgcctgttttttccattcctggcgtcattcctcttgtgccggggctgcccctttatcgggccatgaactatacgatgcaaaacggctacagcatgggtatgcatacctttgtctcggcggcccttgacgcaacggccatcgccatgggtattctccttatctcaggtcttgccaaggtttataagaccagtaaacgccttgtgtcgaaaaaggtccatcatcattccgaacgcttgtaaaaaaacgattgcttataaaccgcgggagtccttccttggagggactctttttgattgggcgccttacgatcttttgccttacagtcttttagcatcacagcatcttagtttccttccattttttcgcccgacagccagaactgaaggctcgtgacagccctttagaacaggcctcgaagaaaacagcttgacagctttcccctttgcgccccgccagaaaaaagattataatatccccaaaaggaggaatgccccatggccatctatcatacgcttacaagaacagtaaaggaaaacccccagacgggaagcatcatcagtgatgccatcatggatccagagaccctcggcaagggggtcaagatgtttgcccaagttacgctgaagccgggctgtgccgttcctattcatcagcatgtcggcaacaatgaaacgtattatctgattcaaggcagcggcgagtatacggacgaagacaaaaaattcaccgtcaaagcaggcgacgtgaccttctgcgctgatggcggcacccatggtctcttgaacacgggaaaggaagacctcgtcttcgtcgcccttattgccaataccatcgcctaaggcatttttgggaaggcggcctggcaggctgctatctctaaaaaatcggagccagctacgccttatcaaagggctgcgggcagcaaagcataaaaagagaagcggaggaagaaatgagcgaatcctttctttccccgtctttttttaaaatagttaatgaaagttttaaataaataagatgaaatatattcaaaacattaataaggaagcgtaacatttttctttcattcttacctaaaaatccccaaaaatgcacagaaaaaccctttctttgcccttctttccagtccgcggtggggatatatgacacactaggaaccgtgtaaacaaagaatggcataaaataaggcgtacagcgcccaaaattttcttgacagggtacgcgaggaatgctagaatagcatggtgtcacaggagaaactatgtcttgacggaggtgcgcaatgtttttggaagaagtaaaaaacggcgctccgactgtggtgggagtaaaacagacccagaaggccctcgtcaaggaaactgcccgcggcgtcatcgtggcgatggatgccagcgatcacatcacaggccccgtcagggccttgtgcgggattcatcaggttcctgtggaaacggtcccgaccatgcaggaattgggcaaggcctgtgggatccacgtgggagcagccgtggcggctgttcttgaaactcggtaaaatcttccgcttgggagattttaatacattaaatcttattcttgagaaaggaggtgccggaatgcctacaatcaatcagttgattcgtaaaggtagacaagttgttactcaaaaatccacggcgccagcattgaaaaattcaccgcaaaaacgcggtgtctgcacgagagtgtacaccaccactccgaagaaacctaactctgctcttcgtaaggttgctcgtgtccgcttgaccaatggtatcgaagttactgcttacattcctggtattggccataacctgcaggagcacagcgtagtcctcatcagaggcggaagagtcaaggaccttcctggtgttcgttatcatatcattcgtggcgcattggatactgccggtgttgctgatcgcgcacaggctcgttccaaatacggcgctaaacgcccgaaagcagctaagaaataagcacaatgcgtgaatgattcataacgaaggaggaattacagatgccgagaaaaggtcctgtaacgaaaagagacgtattaccggatccagtatatggttccaaattattaaccagattcatcaacaagattatgctcgacggcaaaaaaggtgtcgctgaacgcattgtctatgatgcatttgatctgatccattccaaaacgggcaaagaccccatggaagttttcgacgctgccatgaagaacgtcatgcctacgctggaagttaaagctcgccgcgtcggcggtgccaactaccaggttcctgttgaagtccgcgctgaccgcaagaccactctggggattcgctggctcgtcaactattcccgcctgcgtggtgaaaagacgatgtacgaacgtgttgccggcgaactgatggacgctgccaacaacaccggtgcttccgtcaaaaagcgtgaagatacgcacaagatggccgaagccaacagagcgtttgcacattatcgttggtaatctttacaaccaatcattacaaggaggagtaatctgtgggcagagagtttcccttagataaaatcagaaatatcggcatcatggcacacattgatgccggtaaaacgaccaccacggaacgtatcctgttctataccggtaagacccataagatcggcgaagtccacgacggcgccgctaccatggactggatggttcaggaacaggaacgtgggatcacgattacttctgctgcaacgacctgtacctggaaaggacattccatcaacatcattgataccccgggccacgttgacttcacggttgaagttgaacgttccctgcgtgtactcgacggctccgttgccgtattcagtgccaagggcggtgttgaaccccagtccgaaaccgtatggcgtcaggcagaagaatacaatgtaccgcgtatcgcgtacatgaacaagatggatacgacgggcgctgacttcttcaacgctgtccagatgatgaaagatcgtctcggcgcaaatgctgtgcctatccagattccgatgggtgctgaagatcagttcgtcggcctcatcgacctcgtcaagatgcaggctatcgtctatggcgatgaccttggcaaagacgaagaattcgagccgattcctgaagaatacgttgaagaagcccagaaatggcgccagaacctcttggaagccgttgctgaaggcgacgacgacctgatggaaaaataccttgaaggcgaagaccttacggaagatgaaatcaatgccaccatccgtaagatgacctgtgcctgcaagatgttccctgtcacctgcggttcttcctataagaacaaaggcgtgcagccccttctggatgccatcattgcttacatgccggctccgaccgatgtggctgacatcaagggcgtggatcctgaaacgggcgaagaaacaactcgtccttccagcgatgatgctccctttgctgcccttgccttcaagatcgcaaccgacccctatgttggccgcctggctttcttccgtgtctactccggtaaactgacggctggctcctacgtctacaactcttccaaaggcaagagagaacgtatcggccgtatcctgcgcatgcactccaaccatagaacggaaatcgaagaagtctatgctggtgatatcgcagctgccgttggcctgaaagatacgggcacgggcgatacgctctgcgatgaaaaggctccggtcattctcgaatccatggtcttccctgaaccggttatttctgtagccatcgaaccgaagaccaaggccgaccaggaaaagatgggcattgcccttggcaaacttgctgaagaagacccgaccttccgtgtccgcacggatcccgaatcttcccagaccatcatctccggtatgggtgaactccaccttgatatcatcgtcgaccgtctgaagagagaattccacgttgactgcactgtaggcaatccccaggttgcctatcgtgaaaccatccgcaaggctgttaagagcgaaggtaaattcgttcgtcagtccggtggtaaaggtcagtatggtcattgctggcttgaactgactccgctcaaaccgggcgaaggcttcaaatttgaaaacaagattgttggcggtgccattcctaaggaatacattgcacctgttcaggctggtgttgaagaagccatggaaaacggtgttgtggctggctatccgatggttgatatcggtgttaccgtttacgacggttcctaccacgaagtcgactcttctgaaatggcatttaagattgctggttccatgggctttaagtccggtgccgccaaagctgaccctgttctgctcgaaccgtacatgaaagttgaagtgacggtccctgaagaatacatgggcgacgttatcggcgacctgaactcccgccgtggccgcatcgaaggcatggaagcaagaaacggcgcccaaatcatttccgctttcgttcccctgtctgaaatgttcgggtatgcaacggaccttcgttccaagacccagggccgtggcaactactccatggaagtcgaccattacgaagaagttcctaagaacatcgcggaagcgattgttgccaaaaataagggaacggctgaataa

SEQ ID NO: 4 - DNA, 8390 bp, unidentified
  source 1..8390: mol_type = unassigned DNA
atgaacggcaaaccgaacgtcgagcctgcgtggatgtcattgaaacgtctggtcgagacccgtatgatcgagatactccgcaaggaacaggacaacaaaagatacatccggctatacggaacggaagactattggcatgctttcgaagagtcggcctttcagttggagcggctcttccggaaaggagaaaccaccttgttcagacataaggactatccttttccagtggtgatggtcagcattcccgacagcgagttgcgggcttacatccagcagcatattccggtttgtgacaagcacgattacaagaaactgctggcggcagaattctccgccgcagagtatcgtgcatggcatgaacacgcggtgaaagagtttttgtaattctttacgattcccgatgttcgtatcgttttgcggaaattgaggccaaagtagtcacatgcgaccaagcacacgccttgattgatccctggctcacggaagaatcactcaaaaagagcaaggcacagctgattcgcctcttggccgatttcgaccgtcagtttggccgcggaggagagggggggggcatggattcgagatccgagcaggctgttttcggtcttcgaatttcaaacaaataaaatattcatacatcaaatcatgtatcgtttaattgaaaaactgcgactggatcgttatatcagttcacggttgattcttgcattggacctgttggtttcggtcggggcctctatcatctcgctgttagttgcaagcattcttttgggtgccgaagcccttatctggaaaacaatcggctggtggctcggaggatccgttgtcttttcctggatcgcgttcgtgttgctccaaacccacaaatcaatcatccgtcacgccaccttgcggggtttgggccgtttgagtatcgccgttgttttcaagggaattgggatcgcagttatattggctttgggtgtcgctggcccgacatttccagcgatctggatcactctgttgtttgacatattgttgagcctttcaggactggtgattatgcgcgttgccatggttgtcgtctatgactggctcaaagacagccgtcaaaggcattgcaaatgccagcggattctgatctacggaaccggcgacaagggcgtttcgttggtgacccaactccaaaattctcaagagtatcaggtcgtcggtttcctgacctacggcaaaacactgaagaatcatatgctggccgatctcccggtctactacttcgagaccgaagagaatgtcaagtacctgcacaactgcaaggatattgacgccattctcttcgcccacgtccatgaggctcgcgaagagcaggagcgcctaatccattattgtacggactgcaacctcaaggtgctgatcgctccctcgatcgacgaggtggtcgacggcaaggtgcagcgtcaggcaatccgcgagatcaggatcgaggacctgctgggacgtgaagagatcaagatctcgatggacgagatcatcgccaacttccgtggcaagacgatcctggttacgggcgcggcggggtccatcggttcggagttgtgccgccagctggcgacgttcggagtcaaggaactcgtgctgttcgacaactccgagacgccgatgcacaacatccgcctcgagttggaggatcgctaccccaatctgaagttcattcccgtaatcggcgacgtacgcatgattccgcgtctggatttcgtcttccgcacctatcgtccgcaggtggtcttccacgcagcagcctacaaacacgtgccgctgatggaggagaattcctgcgaagccgttctggccaacgttgccggctcgcgcaacgtggcggacaagtgtatcgaatatgacgtcgagaagatggttatgatctcgacggacaaggccgtcaacccgaccaacatcatgggctgtacaaaacgtctggccgagatctacgtgcagtcgctcggtctggccatcgaggccggcaaggtgaagggcaagacgaagttcgtcactacgcgtttcggcaacgtgctgggttcgaacggctcggtgatcccgcgcttccgcgagcagatagccaagggcggtccggttacggttacgcaccccgacatcacgcgcttcttcatgacgattccggaggcctgtcgtctggtaatggaggctgccacgatgtctacgggcacccagatctttgtcttcgacatgggtaagtcggtgaagatcgcccacctggccaggcgcatgatcgaactggccggtctggaggtcgacaaggacatcaagatcgaatacacgggactgcgcccgggcgaaaaactctatgaggaggtgctgtcgaatacggagaacacgcttccgacgtcacacgaccgtatccgcatcgccaaggtccgcgagtacgactacgccgatgctctcaagggtgcgcaggagttggaggagctttgccgcgccgtcgtcattccggacatggttcgcctgatgaagaaaatcgtccccgagttcaagtccaagaactcccgattcgaagagttcgataaagaaacaaagtaaaatcaaccataatgaacacagatttgaacaaagacttaaagattgccgtagccggaaccggctatgttggtttgtcaatcgccacattattggctcagcatcatcaagtaacagcagttgatgttatttctgaaaaggtggagatgctgaatcggaaacagtctccgattcaagacgagtatattgaaaaatatttggccgagaaaccgttgaatcttacagcgacattggatggtgctgcggcctatcggaatgcagattttgtcgtgattgccgctccgacaaactacgaccctgtcaagaactattttgacacaagtcatgtcgaagaggtgatcgatctggtgcttgaggttaatcctcaagctgtcatggttatcaaatcgacgattcctgtaggttactgccggagtttgtatatcaaatacatgcagaagggggtaaaacggttcaatctacttttctctcctgaattcctgcgtgagagcaaagctctctacgacaacctctatccaagtcgcattattgtgggtattccccaaatcatcgacgatgagcggttcaaggaggagaatgatgccattcgtgcaatcgccgatattcaggcgcttaaaaagtcggcccatacgtttgccgctcttctgcaggaaggggccatcaaagagaatatcgacacactgtttatgggcctgaaagaagctgaggccgtgaagctctttgccaatacctatctggcccttcgagtcagctacttcaatgaactggatacttatgcagaggtaaaaggtctggattctcaagccattatcctcggagtaggtctggatccccgtattgggactcattacaacaatccatcgttcggttatggagggtattgcctgccgaaagacactaaacaaatgctggcaaattatgcggatgtccccgagaatctgatcgaggccatcgtcgaatcgaatcgcacccgcaaggatttcattgcggatcaggttctgaaaaaggctggatattatagctactcttcgacaaatgattacaataagaagcgggagaagccaatcgtgatcggtgtatatcgcctgaccatgaaatccaatagtgacaatttccgacagagttcgattcaaggcatcatgaagcgcatcaaggccaagggcgcaacggttatcatttacgagccgaccctcgaggatggatcgacatttttcggcagcaaggtcgtcaacgatctcgagaaattcaagcaaatgagtgctgctatcatagcaaatcgctatgacgcctgcctgggcgatgtcgagacaaaagtctatacccgagatattttcagaagagattaaacagcctattatatgttaccctacaatataatactgaacggcaaaacagttctggtaactggagttgccggctttatcggatcgaatcttgccaaacggcttttgacggactatgcagaactgcaagtcatcggtatagacagcattaccgactattacgatgtctccctgaaacatgatcgactgaaaggcctggaagaatttggagatcgatttgtgttcattcacgactcgatagcaaatcgggaaatcatcatccgcttgtttgacgaatataaacctgctatcgttgtaaacctggcagcacaggcaggcgtcagatacagcattaccaatcctgatgcctacgtcgaaagtaatctcatcggcttttataacattttggaagcctgtcgacacagttacgataatgggcagacaggagttgaacatctggtttacgcctcctcatccagcgtatacggctcgaataaaaaggtaccctattcgaccgatgataaagttgataatccggtatcgttatacgccgcaaccaaaaagtcgaatgaattgttggcgcacgcgtactccaaactatataacataccttcaaccggattacgattctttacggtatatggccccgcaggacgtcctgatatggcctacttcggatttacggacaagcttgtaaaaggagagaagatacgcatctttaattacggaaactgcaagcgggatttcacctatatcgacgatattgtcgagggagtcgttcgggttatgcaacatgcaccggagcgcataactggagaagacggactgccgttgccgccttacaaagtctacaacataggcaacagccagccagagaatctgttggattttgtcgatatcctgcaacaggaattagtacgagccggagtcctgcccgcaaattatgcttttgaagcacacaaagagctcgttccgatgcagccgggagatgttcctgtaacatacgccgatacaacacctcttgagcacgactttgggttcaagccatcgacattccttaggaacggattgagagtatttgcagaatggtatgcaaagtttcacagcgataaataatcactgcctcatatcaacgacatccattctgcatctgcgcgcaatacaaacactttcaaactaaaaaagcacaatacattattttaagcactatgagtattttcaaagacaaaatcctgctaatcacaggcggtaccggaagtttcggcaatgcagttctgcgtcgttttctcgactctgatatccgggaaattcgcatcttctcccgagacgagaagaaacaggatgacatgcggcacttccttcaggcaaatcgtccggacgtttcaaacaaagtcaaattctatatcggaaatgtacgtaaccgcgaggctgtagacttcgcgatgaacggagtagattatgttttctctgcggctgcgctgaagcaggtgccctcttgcgaattctatccgatggaggctgtacgcacgaatgtagaaggcaccaataatgtacttctctccgccatcgctcacggagttaaaaatgtcgtcgtcctctctacggacaaagcagcctacccgatcaatgccatgggtatctccaaagcgatgatggaaaaggtcgccattgctcaaggacgcgctttgggtccggatgcaaaaaccaccatttgctgcacccgttatggaaatgtaatggctagccgtggttccgttattcctctttgggtagagcagatgatggaaggtaagccgatcaccatcacggatcttaacatgacccggttcatgatgacacttgatgacgctgtcgatcttgtcatgtatgcctttgagcatggcaacaacggtgacctcttcgttcaaaaggcgccggccgcaactctcgacgtacttgcagaagccctcaagcaggtttatgccaaggtggatccccaatacggagagaccgaggtgaaaatcatcggtactcgccacggcgagaaactctatgaaacgcttgtcacgcgcgaagagatgcttcgctcggaagattgcggcaactatttccgcattccttgcgacgcccgcgacctcaactacgacaaattcttcacggaaggcgatgatgccatcaaccgcatggaggactatcacagccataacaccaagaggctggatgtcgaaggcatgaaggctcttctcctcaaactcaatttcatccgcgaagacctcggcctgcagcccaaagcaaagtcccgcgattatagaagtgaataacagatgaagattctggtaaccggagcaaagggtttcgtgggtcggaacctttgctcccaacttaataacatcaaaacaggtaaggccaagtgctatggagaccttcagattgatgaagtctttgagtatgacttgaacagcaccatggagcaactggacgcatggtgctccgaatgtgattttgtattcaacctcgctggcgtaaaccggcctcagaacccggaagagttcatggccggcaatttcggctttgcctcaacgcttcttgacacgctgaagaagcatgggaacaaagcccctgtcatgctctcgagttcggcacaggcatctctttccggccgtttcggcaacagcgaatatgggcgcagcaaaaaggcgggcgaagatctctttcttcggtatggagcggaaaacggcataaaggttcttgtttaccggttccccaatctctacggaaaatggtgtcgtcctaactacaacagcgccatcgccacgttctgcaacaacattgccaacgacctgcccatcacggtcaatgatccgaaagtggagctcgaggtcctttacatcgacgaccttgttgaagagatgatcgccgctctgaaaggccaggagcaccattgcgagttcgaagggctgaccgtattgccttctgcggaaggacgttactgctactgtcccgtcactcataaggctacgctgggtgaaatcgttggccttctgcaccagttccgcgacatgccgaagacgctgatgatccccgagatcccggccgacagctttgccaagaggctttacagcacctatttgagctatcttcccaaggataaggccatttttgacctgaagatgaactgcgatgaccgcggctccttcaccgagttggtccacacgctcaactgcggacaggtaagcatcaatatcagcaaaccgggtatcaccaaaggtcagcactggcaccacaccaaatgggagcaattcatcgttgtttccggccacggcctgattcagcagcggaaagagggtacggacgaggtcatcgagttcgaagtctcgggcgacaagattcagagcgtcatcatgctgcccggatatacgcacaacatcataaacctctccaagactcaggatcttgtaactgtcatgtactgcaacgaggttttcaacccggagcggccggacactttcttcgatcccgtagcaagcgaataaaagccgcaagatggctgaataatcaaaacgatctatggaaaccaaaactccgaaattcgattacagtgacattcactggcaaaataacggcaaaatcaaactctgcatcatcgtcggcacccgcccggagatcatccggcttgctgctgtcatctccaaatgccgcaaatatttcgatgtgattctggcccataccggacagaactacgactataacctcaacggcatcttcttccgcgacctgaaactcgccgaaccggaagtctacatggatgcagtcggagatgatctcggcgcgacctgcggcaacatcatcaactgcagctacaagcttttcgctgcaaccaaaccggatgccgtactcgttcttggcgacaccaacagctgtctgagtgtcatcggggcaaaacggctccacattccgatttttcacatggaggccggcaaccgctgcaaagacgagtgcctgccggaggagacgaaccgccgcatcgtggacatcatctcggacgtcaatctcgcctactccgagcatgcaagaagataccttgctgaatgcggacttccccgtgagcgtacatacgtcaccggttctcccatggccgaggtgctccataagaaccttgccgagatccaggcatccgatgtacacgagagactcggactcaagaaggggcactacatcctgttgagtgctcaccgcgaagagaacatcgacacggagaagaacttcctctgccttttcaatgcgataaacgcaatggccgagaagtatgacatgccgattctctacagttgccatccccggagccgtcaccgccttgaggctacaggtttcaaacttgatccgcgagtacaggtccagcagccactcgggtttcacgactacaattgccttcagatgaacgcattcgctgtcgtttccgattcgggcacccttcccgaggagagttctttcttcaccagcgtaggacatccttttccggca

SEQ ID NO: 5 - DNA, 11582 bp, unidentified
  source 1..11582: mol_type = unassigned DNA
  misc_difference 568: note = a, c, g, or t
  misc_difference 569: note = a, c, g, or t
  misc_difference 570: note = a, c, g, or t
  misc_difference 571: note = a, c, g, or t
  misc_difference 572: note = a, c, g, or t
  misc_difference 573: note = a, c, g, or t
  misc_difference 574: note = a, c, g, or t
  misc_difference 575: note = a, c, g, or t
  misc_difference 576: note = a, c, g, or t
  misc_difference 577: note = a, c, g, or t
  misc_difference 6182: note = a, c, g, or t
  misc_difference 6183: note = a, c, g, or t
  misc_difference 6184: note = a, c, g, or t
  misc_difference 6185: note = a, c, g, or t
  misc_difference 6186: note = a, c, g, or t
  misc_difference 6187: note = a, c, g, or t
  misc_difference 6188: note = a, c, g, or t
  misc_difference 6189: note = a, c, g, or t
  misc_difference 6190: note = a, c, g, or t
  misc_difference 6191: note = a, c, g, or t
  misc_difference 6566: note = a, c, g, or t
  misc_difference 6567: note = a, c, g, or t
  misc_difference 6568: note = a, c, g, or t
  misc_difference 6569: note = a, c, g, or t
  misc_difference 6570: note = a, c, g, or t
  misc_difference 6571: note = a, c, g, or t
  misc_difference 6572: note = a, c, g, or t
  misc_difference 6573: note = a, c, g, or t
  misc_difference 6574: note = a, c, g, or t
  misc_difference 6575: note = a, c, g, or t
  misc_difference 7329: note = a, c, g, or t
  misc_difference 7330: note = a, c, g, or t
  misc_difference 7331: note = a, c, g, or t
  misc_difference 7332: note = a, c, g, or t
  misc_difference 7333: note = a, c, g, or t
  misc_difference 7334: note = a, c, g, or t
  misc_difference 7335: note = a, c, g, or t
  misc_difference 7336: note = a, c, g, or t
  misc_difference 7337: note = a, c, g, or t
  misc_difference 7338: note = a, c, g, or t
tcagttgtccgtgtcgttcggcatgccggcggccgagtagagccggagcaggtcgtaggcttcgccgatgccgagttcgcccgccttgctgatgtcgagcagccccttgcgggtatccttcatgtatatctgtaccaagcccctgttgaaataggcttcggcgaggcccgggtcgagggcgatagcgtgcgaatactcctcgaatgcctccgggaagcgtcccgacagggcgaacagattgccccggttgtagtagatgcaggggaggttcggattgagtttggccgccttgttcaggtcggagatggcttcgtcgtagtcgtacgtccgtgccgcgtcgcgcatcctggccgaacggtcgctttcgggcgaaatgcgcgaataaccgttgtccatggaggagatgaaatccgtcatttcggcctgtacggcgctgcgggcgatgtagaggaacgggttgcgcggagagagcgtgatggcagtcgaaagggctccgaccgcgttggtgtactgtttcagcagcgcctgtgtcatgccccgcatgaaataggcctgccagcggctgccnnnnnnnnnntccttcctgacgagggcggcggccgccatgtcgcttccttcgcatccgaagacgaccgatgcgtcgcccagcgaagcgatgaactcttccgcctcggcctctccggggtgcaggatgaggccccggcctggctccgcatccgaacggagggtgaagccgtaggaggggcgcagcgttattcgctctttcgttcccattgccgtattgcgtaccggtccgtcggagagctgcgtgtcgaacgacagcagtttgtcgaattgccgggaggtgtcggcaaaggagacttcgctttccccttcgagccgtgcccggtattcggcgattttgcgttcggcgacactgcggtcccgttcggcgccttcgctgtcacggagcaggtatttgaccgaagcccggccgaggtaggcgttggcgaagtcgggatagagttcgatggcgcggtcgtagtcttccagcgccccctgcaattcgccgagtctgaagcggaggccggcccggttgtagtagacgatgacgttgcccggcgagtaggcggctacctggtcgtaatcctccagggcccggttgtagtcacccgtttcggtgcgcaccagcgcccggttgaagtaggttaccgaactggtcgggtcgagctccaatacccggtcgaaatcggcgagtgcggcctgcaggttgtgcttcctgtaatagaccatggcccggctgaagtaggccggtatgtacgacgtgtcgcaccgtacggccgtatcgaaatcggcgagcgcttcgtcgtacctgccctgctccatgtagagcgaaccgcgttcgaggtaaccgcgcggatactcccgattggtgcggatggccctgccgaagtcctcgtatgcggcgaccgtatcctgcaggtggaggtacgctgtgccccggttggcgtaggcgtcggcgtcgcgctggttgaagcgcatgtacatgttgaagtcatcgacggcttcgcggtagcgccccgtgaggatgttcgtgacgccccggctgtaatagggggccggataatcgggacgcagttcgatggcttcgtcgaagtctttcatcgcgtcgtcgtagttgccgagccgcgaacgggtgagtgcacggaattggtaggcgttggtgtaaaccggattttttgcgagtgtcagggtgaaatcccgttcggcgcccagcaggtcgtcgaggtgatacttcgcgataccgcgccagaagtaaccttcgtaggcgtccgggtcggcgcgcagcagaatgttgagcgtctctatcgcttcccggtagcggctgtccataaccatgtcgcggcctacccacacgaaatactgtttgttgacctgcgctccggcgttgtctgcgaacatgcagaaaaggaatagtatggccccaatgaatttacgcattcccggattttaagcttgctaatataacgattataatcctaatacggatgcgccggaggcgttttcggagcggcagcggtctgagaggacttgttaagtggctggatgttagtgtattcgtatccgatggacggtcggccgacgggaaagccccgaaaaaacgggacgtttatgcacctttctcggaaaaaagagtaaatttgcatccggccctaatttggaacggtgtatttgcagctatcccggctatgtagagatagtattgtcatacagtatgttgcaatgcaaaggcaggatgccgggccgtcttgcagggccgtggcggggcatgtcggcggcggtgtcggcaggagctttcggaaatcggtgatattaaaaggaatcccgtgcggcgcagcccgcacgcgggtcggttactaaataaaagttaagctaatgggaaaacagttgaaagtagttgtactcgccaagcaggttcccgacacgcgcaacgtgggcaaggatgccatgaaggccgacggtacggtgaaccgtgccgccctgccggccatcttcaaccccgaagacctgaatgcgctcgaacaggcactgaccatcaaggacatgtgcgaagggagtacggtatatatcctgaccatggggccgctgcgtgctgcggatatcatacgcgatgccatgttccgcggtgccgacggcggataccttcttacggacagggcattcgccggtgcggataccctcgccacctcctatgccctttcgcgggcgctggccgagataaagcccgatatcatcgtggcgggccggcaggccatcgacggcgataccgcgcaggtggggccgcaggtggctgagaaactcggtcttccgcaggtgacctatgcggaacaaatcgtgtcgatagacgacagccgcatcgtcatcaaacgtcgtctggaacatggcgtggagaccgtttcgtcgccttatccgatgctgatgacggtgaacggttcggctcccgaatgccgtccccagaacgcgaaacgggtgatgaagtacaagcatgcccgtacccgcagcgaactgcaggagctcggcgaggattactacatgcagctcctcgaagccaagagttacctgaaaatcaatgagtggaacgtcggcgatatcgatgcggacccccagcagctcggactgctcggttcgcccaccaaggtaaaacagatagagaacgtggtattccaggccaaggagtcgcgcagactgaccgcttcggacggggatatcgattcactgatggtcgaacttatttccagccacacgctcggatagaggaggaatgtaagatgaacaacgtattcgtatatattgaaacggaaggcggcaaggtggctgatgtcagcctggagctgctcaccaaaggccgcgaactggccgaccgcctcgggtgcaaactggaagcgctcgccctcggtcacgaactcaaggagatggataagattgtcggacgctacggagccgatacgctctggctggcggacgatgcggagctcgctccgtaccgcaccctgccccatacggcggtggtttgcggtatcttcgaagaggagaaaccccagatagccctgttcggtgcgacgccggtggggcgcgacctcgctccgcgcgtctcctccacgctgcgcagcgggctcacggccgactgtacgagcctcgtcataggcgaccataccgatgccaagagcggccggtcgtacgagaacctgctctatcagattcgtcctgccttcggcggcaacatcatcgccaccatcgtcaatcccgaccatcgtccgcaaatggcgaccgtccgcgagggcgtgatgcgcaaggaagagctcaaggagccgaagcatccggaggtgaaacgggtggacgtggcgaagttcgtgaaggagagcgacctcgtcgtgaaaataatcgaccggcatatcgaggagcggggcatcgacatcaagaacgccccggttatcgtggccggcggttacggcatggggtcgaaggagaatttcgccatgctgcatgaactggccgacctgctcggcggcgaggtgggcgcgtcgcgtgccgccgtggatgcgggatttaccgaccatgcccgtcaggtggggcagaccggcgtgacggttcggccgaaactctacatcgcctgcggcatctccggtcagatacagcatacggcgggaatggaccaggcctccatcgtgatagccatcaataccgacccggaggcgcccatcaacaaattcgccgattatgtgattaccggcagtgccctggaggtcgtgcccaaaatgataaagttctataaacaaaactccaaataggcgggagccgtttccggggagggccgagagagacgccgcttcccgtatccgggaacctgccgcaaggcaagatatgggaaacttttttttagacaataaagacctgcagttccacctgaatcatccgctgatgaagaaaatcgtggagctgcgcgagaagaatttcgccgacaaggatgtgtacgactatgcaccggcggatttcgaggatgcgatggacagctaccgccgcgtattggagataacgggcggaatatgcgccgacgtgatagcggccaatgccgaggatgtcgataaggagggaccgcgggtggtggatgaccatgtcgtctatgcacgcggtacgcaggctaacctcgatgccatccgcaaggccggattgggcggtatccacctgccccgcaagtacgaaggactcaacatgtcgctcatctgcttcgtgatggccaacgagatggtggcccgcggcgatgccggtttcgagaatatctggggattgcaggactgcgccgaaaccatcaacgagttcgccagcgaggagataaagcagaaatatctgccgctcgtatcgcggggggctacctgcgccatggcgcttaccgagcccgatgccggttccgacctgcaggcggtgatgcttcgggcgcattacgacgaggccagcggcaagtggttgctcaacggcgtgaagcggttcatcaccaacggcgacggcgacatctcgctcgtgctcgcccgctcggaagaggggacgaaggacggacgcggtctctccatgttcgtctatgaccgccgcgacggcggagtgaaggtgcgtcgcatcgagaacaagctgggtatcaaaggctcgcctacctgcgagctcgtctatacgaacgctccggcagagctcgtcggcgaccgcaagctcggtcttatcaaatacgtgatgtccctgatgaatgccgcccgtctcggtatcggcgcacagggcacggggctgtgcgaggccgcctaccgcgaagcgctgaagtatgccaacgaacgccagcagttcggcaagcccatcatccagttcccggccatttatgaaatcctgacgaacatgaaggcgaaactgtacgcatcgcgtgcgatgctgtacgagacggcccgtttcgtcgagatatacaagtgctgggcggagataggcaaggaacgtccgctgacggccgaggaaaagagcgagatgaaggcctataacaagctcgccgatgccttcacgccgctctgcaagatgttcgccagtgaatatgccaaccagcttgcctacgaggccatacagatacacggcggttcgggctacatgaaggactatcccatcgaacggatttaccgcgatgcccgcatcaccaacatctacgaaggtacgacccagcttcaggtggtggccgccatccgccacgtgacgaccggcacgttcctcgcgcagatgaaggcatacgaggctgccgagcgcccggccgagttcaagttccagctcgacaagctcatcgccatgcgcgaggagtacgaacgggcgatagcccgcgtaacgggcggcgacagcgaacagatagagttccatgcacgtcggctcgtggagatgaccgggcacatcgtcctttcgcacctactgctccgtcaggcggccgagagcgaagagtatcgggtgcctgccgaggcgtacatcaagtatgcccaagcgcagaacaaggcggcggccgaattcatcgccaactcctgctgcggcgatatcgacctctacaaggcggttctgaacgagacgctggaataagataagaacggttgcgcggaaagcgtagccgattgggacgaggggtgcggcaagattgnnnnnnnnnnccctacacgacgctcttccgatctacccctcgtctcgtctttgctccgtggacagccgtaacggattgcctgcgaaacggtgcggtaaagtgggtgtcgttgtcgggcagtgtatcccggccggtgaaacccagccggacatgacgctgtttcgggaagacgttcggcggcggtgacggaatcgggagttgtctcggtcggagctgagccccttgcctgcgtgtggttgttgggcgatttccgtgcagaacctgtatgcggttcagtcggaatgccggaaagcggcgacagggaaatggccggtcggttttcggaatctctgtggccgtactgttgcgggggcaggaacagtacggcacggaatgctgccggttnnnnnnnnnncgatgtccgggaaaggatggggagaggctggcaggtcagagttcctctatgaaatgcggatatttgttccggtattcgtcataaatccgttcttcctgtccgtcgaggttgagttgatacatgcgaaagacgaccgggatgtttttgggctgccagagctcccggtaggagtaacggtatttcatacctatggcctgcatcacgtttccgcttctcgggttgttcacgtcgtgcgtcgccgtaacataagggagaccgtctcgccgaagccggtcgatgatggcctggcaggcttccgtacacaggccccggtgccagaatgcttttcggagtccgtaccccaggtcgtggctgccggcgccgcttacgtggacataaccgacgggtacatcgctgcctttcatgcagacggcatcgtataaaagcgattcgccggaacgcagtgctgcaagtttcttgtgcaggtatgcctcggcctcttccaaatgcttcagcggaaacatgggcaggaatgtattcacttcggtgtcgcccagaatgtccagcagggctttcgcatcctgttcggcgaacggccgcaggtgcagccgttccgttcttatgtcatgggacatgcgttcgttttgtgagggttgtcgcattgtatccgggcatttatcggtgaggcggtgccatgccccgtacttttgccggaaaggcgggaacgacggattcggggaaattggccggaggacgtccgttcgctgccgcacgnnnnnnnnnntttccctacacgacgctcttccgatctatagaagaggtcgctgatgatgctgttggagaccatgagcttttcgggtggtatcctgtacacattgccttcgcggactatcaagccgtttcggagaaacttgccggcacaatattcgaagtagagcagtcccttgaacccgaaccgttcggagagcgcgtcgcgtcgaataccgtaatcggtacggagggcggtcatgatatattcgttgtacttgtctatgcgggagaggatttcccgtccgtatatccggtcggtaccggccagttcgatgtacttttcgaggttgttgaccgagtagatacgttggtccccgtcgtaggagtgggccgatgggccgaggccgagataggttttgccgcaccagtaggcccagttgtgcagcgagcggaactcttcgccgcgggcatagttggagatttcgtagtgacagaaaccgctgtcggtcagaatgcggtggcatatcatgaactgctcctcacatatttcgtcgctggcaggtatgagcgttcccgcctccgccatgtcgccgaagacggtgttgtcggagatggtcaggcagtaggccgagatgtgctgtacgccgagtattacggcacgcaggacattgttttcccactcgcggagcgacatgccggggatgccgaagatgaggtcgagcgaaatgttgccgaacccctttgcctgggccgcccggatagcctctaccgcctgttgggcgttgtgccgtctgccgagcagcttgaggtcgcggtcgatgaaggactgtacgccgaaactgagccggttgatgtcggtacgggcgagttcgtccagatagtgttcggtgatgtcgtcgggattcgcctcgaccgtgatttccgataccccttccagattccagagcgagcgggccttgtcgatgagccgctggagcagggcgggcggaagaagcgacggggtacccccgccgaagaataccgtgtctaccggggtgtcgccgaaatagccgcgccggtactccatctccctttcgagggcttctatgtaatcctccgttttggcacaggatgtgcttttataaaaatcgcaatagttgcagcgcgttttgcagaacgggatatggatgtacaatgcggccatttctgggtggaatgatggtgtgtttcgatttcgtaaggtgctgaacacaaaaaacgtgcgaaagttagcaaaaaaaccgtaataatggccgccgctgccgtcattatgcggtgggggaagatgtgcagataggtattcccggtttccggtacggtaaaaaaagtcgattttatacggtggaattagtaccgcaagacggaattattccatatctttgcgccgttgttagtgtgctaacaataattccggcgggaaagagttatataaccggattcgcagaccggaccgttccgggtgtccggaggcgcatataacaggtgagaatagcataaacacataatatacagaagattatggcaaagaagattgatttgaccaagtatggtatcaccggaaccccggagatactgtacaatccgtcgtacgaacggcttttcgaggaggagatgaacccggccctcaccggttacgaacggggccaactgaccgataccggtgcggtgaatgtgatgaccggcgaatataccggccgttcgcccaaggacaagttctttgtgatggacgacacgacgcgcgataccatctggtggaccagcgacgagtataagaacgacaacaagcccgtgagcgaggaggcgtggaaggagctcaagaagattgccacgaacgagctctccaacaagaagctcttcgtgatggatactttctgcggcgccaatgcgaatacccgcctcaagatacgtttcatcatggaggtggcatggcaggcccacttcgtgaagaacatgttcatccgtccgaccgaagaggagctggagaactacggcgaacccgacttcgtggtgatgaacgcttccaaggcgaaggtaacgaactataaggagctcggcctcaattccgaaacggccgtcgtcttcaacctgaccgaaaaggtgcaggtcatcatcaatacgtggtatggcggcgagatgaagaagggcatgttctcctacatgaactatctcctgccactcaagggcatcgcttcgatgcactgttcggccaacaccgacaaggagggcaagaatacggccatcttcttcggcctctcgggtacgggcaagaccaccctttcgaccgacccgaaacgcctgctcatcggcgacgacgagcacggctgggacgaccagggcgtgttcaacttcgagggcggctgctacgccaaggtcatcaacctcgacaaggagagcgagccggatatctggcacgccatcaagcgcgatgcgctgcttgagaacgtgacggtggacaaggaggggcatatcgacttcgccgacaagagcgtgaccgagaatacccgcgtgtcctatccgatatacttcatcaacaatatcgtgaagcccgtatccaagggcccggctgcggagcgtgttatcttcctgtcggcggatgcgttcggtgtgctgcctcccgtttcgatcctcactccggagcagacgaaatactacttcctctccggttttaccgcgaagctggcgggtaccgagcgcggtatcaccgagccgaccccgactttctcggcatgtttcggtgcggcgttcctctcgcttcaccccaccaagtacggcgaagagctcgtaaaacgcatggagaaatcgggtgcgaaggcctacctcgtgaacaccggctggaacggtacgggcaaacgtatctccatcagggatacacgcggcatcatcgacgccatcctcgacggctcgatagacaaggccccgaccaagcagattccttatttcgactttacggtgccgaccgcactcccgggcgtagaccctgcaattctcgacccgcgcgatacttatgccgacgcggccgagtggaacaggagagcggaagaccttgccggccggttcatcaagaacttcgagaagtttaccggcaatgcggagggcaaatcgcttgtcgccgcaggtccgaagctgaaataagagtgtagtgttgtcactcatcgaacggcggggcggcgtaatgtcgtcccgcttttttcaaaaggagaagagatgaagacgattcgaaggtacatgtttttcgctctccttctgctggcggcagtcgcctgccggccgggagcaggggcggaacggggagcggccggattggattacggtcgggccgacatgtggtatcggggcggtacggcggcttcggcggagaatgccgatgtgttttacatcgccccgacctgcaattggggacggacggatgccgacggcaccgtgcattggcagatggatgtgtacgataccgtgcagcgtgccaatctgcaggcgccgctcagactggccgagcgcgtgttcgccgaaggcaaccgcttctacgcgccttattaccggcagattaccatggagtcgtggttcgagcccgattcggttatcggggcaaggtttgccgtggcgatggaagatgtacgggaggctttccgttactatctggagcacttcaacgggggccggcctttcgtattggcgggacacagccagggagcccggtgcgtgttggagctgttgaaggaggagatgaacgacagtctttacggccggttggtggccgcttacgtggtcgagtaccggattaccgacggagagttggagggagcgcggtatgtcgtgcctgccgccgactcgctcgatacaggtgtcgtgatatgtatcaacagtgtggcgaatacctctgcgctctctccgctctttcggggaaaccgtgcctgcatcaacccgatgaactggcggacggatgctcttccctcccggccggaggagaacctcggtacgcttttcctcaatccggacgggacggtcgcccggcaggtggaacccgtgacggcacgtatcgatacggcctcctgtacgctcatcgtggacggtcttgttccggaggagcatttcattccggccatagcccccgtagcaccgctgggaaattaccatgtgcaggaactgaacatctatttcggcaacctgcggcacaatgtggcgcgaagggtgcgggcgtattatgaagaaggccgtgcggcacgtttcccgaccgcatgccgggctgccgcagtccgggaatga

SEQ ID NO: 6 - DNA, 8401 bp, unidentified
  source 1..8401: mol_type = unassigned DNA
ctacttctctattcttatccagaaatcatcctctgccatacgcccctgcgtcttttctattatcataaatgtgtcataaaacggaagtccgatatcctccagcacaagcttcatcttatctcttgttcttggaaaacatcttgactcaagaaattccttatagtcattgtaatcaggattttcattaacgccaaatgcacgaaacatcagcttgtctgtataattctttattctcacaagctgctttgcgtcatcaacatctatcagtgtacagatgtcatccttatacatataaaacattctgacaggaaattctttctccggtattatcagtctgttcacataatcaggataatcattaatcatcttagaaagtaaaacaattggacctgtaatcttagcattttctttttcccatcgttctattgtcggtttcgatgttcctacaagactggcaaattctttctgtgtcagtccaagattctttcgcagattcttaatatcctcagatgtaatataatcttttaatgcaaacattgtgtcacttcctttcagtccttgataaataatgacttttcataacccaagtgtaccatgtacacattttattgtaaacaaaaagtcgttaaaatgatggtttttatttataataatctcatcattttaacgactttattcatataaaacatttcaatttcttaattatccagatttattggtaactttaattctctattagaattatatagcctccagcgcctgtgcaatatccgcaatcagatccttcttatcctcaagaccacatgagatacggataagtcctgctggaattccagcggctttaagctgctcatcattcatctgtctgtgtgttgatgttgcagggttaaggcagcatgttctcgcatctgctacatgagtttcgatagctgcaagctttaaattcttcataaacttctctgcagcctctcttccaccctttaactcaaatgaaactactccgcagccgccatctggcatgtacttcttagctgtctcatagtacttgttagaaggaagtcccggataattaacgaattcaaccttatcctgcttctcaagaaattctgcaacagcctgtccgttttcaacatgtcttggcatacgcacatgaagtgattcaagaccaagatttaatataaatgcattctgtggagactgtatacagcctaaatctctcataagctgcgaagtacattttgttataaatgcgccttctttaccgaacttctcggcatatgtaattccatgatatgattcatcaggtgtgcagagtccagggaatttgtctgcatgtgccatccagtcaaactttccgctgtctacaatagctccaccgactgctgctccatgtccgtccatatatttagttgttgaatgtgttacaatatctgctccccattcaattggtctgcaattaactggtgttgggaatgtattatcaacgataagtggtacaccgtgtgcatgtgctgcatttgcaaacttctcaatatctaatactgtaagtgcaggattggcaattgtctcaccaaatactgctcttgtgttgtccttgaacgctgcattaagttcttcctctgtgcagtcagggtcaacaaatgttgcttcaattcccatcttcttcaaagtaacatcaataaggttaaatgttcctccatatatagaagatgatgctactatatggcttccagcttcgcaaatattaaataaagcgaagaaattagctgcctgtcctgatgaagtaagcattgctgcacttccaccttcaagggctgcaatcttagctgccacataatcattagttggattctgaagtcttgtgtagaaatatccacttgcttcaaggtcaaaaagttttcccatatcctcacttgtatcatacttaaatgtagtagactggattataggaatctgtcttggttcaccatttccaggagtatagcctgcctggacgcattttgtgttaattgaataattatccatatttccctccggctttatatgtttacatgacatgaattatatcacaaactcaaagcatataacagcacattttagaaatcgggctacacatttattaacataatatgttatgatatcaattatgctattatcagtttttaataatattgtataataattaagaggttttattatgaataatcagtcagcgactatgaacacaaaagataatccgcttggatataaaaaagaatccactctgcttgttggttttgcaattccatgcattatttcaatgcttgtgacagcgctttacaatattgtcgaccagatatttatcggtcagggaatcggcatgcttggaaatgctgcgactaatattgcatttccactttcaacaacatgtacagctatatcgcttcttctcggtataggaagcgcgactaatttctcgctgtatcttggtgccggtgaaaagaacgaatctgaaaaatatgccggcaatggtattgtgcttatggcattgtttggaatatttctgttccttgtaaccactatattcttaacacctatgcttaaattctttggtgcaactaaagatgtacttccttatgcaaaggcatatacaagaatcactgcgtttggtttcccattcttaattgcaaacactggtatgagcaagcttattcttgcagacggaagcccgcgatattccatgatatctatgcttgctggtgccatagtaaatactattcttgacccattgtttatctttgtctttaacatgggaatgactggtgctgctcttgcaacaattacaggtcagataatttcattttcaatcagcctgcgttatatgtttcattttaaaacaattaaacttagcagggaaagttttattgtttctgctgcccactgtaagaaaatatttattcttggagcaagtgcctgctttaaccagattgcaatgaccgttgtacagattgttatgaacaatacgctgtcacactacggcgcacagtctgtatatggcggtgacattcctcttgcctgcgccggaattataactaaggttaatatgattttcatgtcatttgttatcggaatatcccagggaacacagcctgttataggttttaactatggtgcaaagaagtacgcccgtgtaagaaagacttatcttcttgcacttggtgcagcttctgccctgtctttaatcgcatttgcatgcttccagatattcccaagacagattatcagcatatttggtaacggaagtgacctgtactttaaattctctgaaagatatttcagaatatatatgtttctgacaattgttaatggcatccagcctgtaacttccaatttctttaactcaattggcaaatcacagcttggtgtatttatgtcgcttacaaggcagattattttcctgctgccacttatcattatatttcctatattcatgggaattgacggtgttatgtatgccggacctatagcagacgctgccgctgctatcgtatgcggatactttactataagagaattaaaagaattaaaaaagctggaaatcgatttaactaaatgttaaaatataagttatgcgtgtattcgtatatttaacggattatttttaaaaggatttttaacatggataattttaatgctcgatatgaaaaagaacttgcctatcaggaaattacctcgaataaatacactctttacggtattctgctatttataggagttgaattactgatatggcttcttaatataattggattttttgaacttgataaccagataatgtctgctgttataggttcatctattgtattatttatccccataatccttatcatgataaaaggagatttatctaagcctgcctacaaatacattgccatgactcagatatgtataattacaggaactataattacatttttatcatatcacgctattcttctgtatgttcttccgcttttatttgctggacattacagaaaacgttctgtgctatggtacacatatgtattaagtgtattaatgctttttgtaagttcgattcttaattattattacggaataatggataccaatctgcttattgccggaacacaccagcgcaaatggtatcttgaccttatagcaaacggaggaagctttacatataatgcaaatgctgtatttataatactaatctttgcatgtataccccgcagtatgatactgcttgtatttacattcatgatacagtatatagttaccgccagcctgcatgatgccgcaagaattgcccagcttacatatcttaaagaaaccgattctttaaccaggcttttcaacaagaataaatacaacgaaatggtatcaatgtattatcctaatgtaaataatattactgttattttctgggatcttaacaacctgaaaattattaatgacacttatggacatgaatatggtgataaagcactttccgcactagcttctgtattatacgcccattcagatgaacaatgccgccgtgtttacagatttggtggtgatgaatttgtcatgataattgataacccacatgacggtgaagctgaactaataatatctgctgttaaagatgaacttgtgaaatgcagttatgatatttcaatagaaatatcaagtgctgtcggtgttgcatccggcaacggaagtgatattattgaaatagtaaaaaatgctgactcagcaatgtacgctgacaaacagctctaccaccatcggtgattgtgaaatactgtatatgcgttgatttatgccgccagcttaactgctggcggttttttctgatttttggggatgggtggtaaatcatggctttatgagaaaaggcttcctataagaaaataaatatatctcatcttataggctgattttccctcttacgctgtctaaaatatggttttgaagttgaatttcctataatgttcatgattatagttactttataggtgcaatttacaagtaacatagtagtcgatgtgatttttgacctataaaggctttagatttacgactactgataggtatcatatgtttttcaatcaacagtcttaatatatcttcttctttttgttgttccaatacaggctaataactccttattcactaattcatttaatagttgtcttgttctatgaccttttaatccaatcttctctgctatttgttccgtactatattcaacacctatatccatatttgttaaaatctgtttgtgtcttgctgataaatctgacctattcatgttttttgaatctgctgatttttctgctaattcttcatcagattccttttccatcttcatcacaagcattatgaatttaataatgtatagggaagtaaaaacttcctcgtgtatcaaccacgaaaaagggtggtacccaaaagtaccaccctacagactataaatctatcctacatactctaaaaccgactattttctgccaagaatcttagacattcctgttgccacaaaaagatatacaaccatattaactactgatttaagaagaagtcctggtgccgaagctaatcctgctgcaaagctgcctgctattattgcaccaaatacaacatatcctaatgtcatgaaaagtaatgtaacaactgcaccaacaattccttttactgaaataaccttacatctgtcaaacttacttttcatattgccaaatattgcacatgctattattggcatgattgtctttattataagtgttggaactgcccatactgcaaatccagttaatatatctgccatcgcagaaccaattcctcctgcgagtgcgccaacaactggtccgaagaaaaatgcacatataagtattacgctgtctcccaaatgtgcatatcccaaaggaataggaatctgtaccaccattgttgcaacacatgtaagtgccataaatactgctgttatacacatctgtgttacagtaatatgaaatccttctttcttaatcccctgctgtactaactgtgtctgttgtgcttccattttatctgcctcctatatcaacatactttaattgcacataagaataacagataaagtgacatgtgtacattgtcattttgagttaaatttatatggtcagatttaaaatctattctccctcttctttcttaatcaattcatccataagttctttataatctgaggcttcaagcggaagttctacttttatgcctttgcatgctgaataataagcactggcacacaacatgagtgaattaatcccctcactgcccttaactgtcagataagactcatcacctttaagcaccgcatttgcaaaattttcaaataactgtgcatatggctcagatgccttctcgaactgtatagtttcctctgtaaatgtcatattctgccttgagttgacttcttcgttcttgatatactcacatacatctctgtgtcttgtaatggtgagcgtgtcgtcttccagaagaattcttgccttcgttcctataatctcaagtcgctcttcgtgacaggcttcgcctgtggatagtataaatgtgcctgtctttccattgtcgtattccataacgatagtagcctcatcatctactataaaatcgttgtattttccaaatggaatcgctgcaaaaagtttctgtggcattccaaacaggtactgatacatatcaagaatatgctgtccctgattgataagtgctccaccgccttcacctgcaaagctgcttctccatgagccagatttatgatagtatgaggttcttaaatatcgtgaattaataagacatactctctttatatctcctaattcaccattatctatcatctgcttaatcctgatgtatttagggtaaagcctctgatgaaatatcattccatatattctgccactttcactggcagccttctgcattgcaagtgcttcacctatatctgccgctgccggcttatcacatagcactgcttttccctttgcaaatgctttcaaagccagttctttatgagttttatgaggagttgctataataactgcatcataacatgattctcctgtctcaaataattcttctgcactcctgaatatccttgcattatcaccatcagtattaacaagtgactcgccccattcaataagttcatctcttcttataaccacagctgataatttcatatgtggaacttctcccgctgttatcattctggcatatttctttccaacacttcctacacctataagtgccacctttaacaattctttctgcataccatgtaccccgtttcattatattaactatgcaccacttctcctgtttccacagccttcttcagtaagatggcggcatacgcatctgcaagggcatcctccatactatatggcgcagacgcatttcctctgctatactctgccgtattcttcatcatcactgcgattgctgtttcatcttctgacagaccacacaatccaaattccggctcatatataattttattattaaatgaaattttcttaatttcacgaatcttctcaaaattaggattagagtttcctgtattaatcacatgggaatccgttatgattctgccagtctgaccttcattattctcatcaagataatatacacattcatcaataagttcgcctcttaccccctgaattttaagcgtattctttctgattggtgacctgtactgctccgaatcaaaatcataccatgccactttcccgctttcgaattcaaatgtggcaacgcatcttttctttcctgcaatcctgccatcagtgaacttatcatatcttgtaagagtctgcgttgtcgggaattcgtatgtcttggctgaaactgtatacttctctcccggcttaacaccaagaaaagctcttataagacttattccatgatattcatgcgccagtgacacatttgcacagctgacatcaccaataattccttcattaataagctttaatgatgctttaatattaggatactccctgtactgttcagcaaccacctgttttttaccacatttgtaataagaatacagtttattaagcgaatccgtatcaagtgccgccggagtctctgataaaacagttattccacggtcaagccattccattgaaacatcacatatagcgtccttcttaacagccacaactgcaaaatccggtttatatgccacgcattcttctatagatgtagttgtatgtatctgatactgaccggcaattaagtccgctttctcctgcgtcctgcaatacatcgcatcaagactgaatatttctggcagagcctttgctacgcgtacataatataaggcccgccagcctgagccgattataataaaatgatattttgccat

SEQ ID NO: 7 - DNA, 7577 bp, unidentified
  source 1..7577: mol_type = unassigned DNA
atgcaaatggcagtaattgaactggaaaaaaacatttcatatatagagccgacgcaaaatccattgtcggcaaatgtcgtgctgattgaaggagaaaataatctgtggctgtacgatgtcggaaataatccgtgtgtgccacagattatatctgcttttaacacttcaaataagaagataaatgtgattttgtctcattttcatgcagaccatataggtaatcttgataaaatcagctattcaaatgtgtatcagggcagattgacttataaatatacgaaaacaggtgaaatactggaaaatgatttgtatttttgtgatggtaatatgaatcttcatatttttccactcccgtcaagtcatgccaaaggttctttggcattagaaataaatgaaaaatactgttttttgggggatggtgtttatgcaatgcaaaaaggtgaaaaaaggctttataatgcaggaattcttaaagaagaaattgaaattctaaaaaatataaaagcacaacattttatgcttagccacagaaatccttttgataattcaaaggattcggttataaggctgcttgatgggatatatgccaaaagaaaaagcggagagccgtatatttccggcattgacggggaatgagaggtattaagttagtcgtatcagaaagtatgcagagtatatttatgtaatctgcacactttacatttacacaaaataggtatactatataattaaaattccactatatacaggaggagaattatgaattattctattaatacaaaatgcgtacaggcaggttatacaccgggcaatggcgaaccaagacagattcctattatccagtcaacaacatttaagtatgatacaagcgaagatatgggaaagctttttgaccttgaggcatcaggatatttttatacaagattgcagaatccgaccaatgattatgtagcttcaaagattgcagcacttgagggtggtacagcggcaatgcttacttcatcaggtcaggcagccaatttttttgcgttgtttaatatatgcgaagcaggaagccatattgtttcttcatcatctatttatggtggaacatttaatcttattgaagtaactatgaaaaaaatgggcatagatgtgacatttgtagaccctgattgcacagatgaggaacttaatgctgcatttaaggataatacaagagctgtttttggagagtcaatagccaatccggcacttacagttcttgatatagagaaatttgcaaaagcagcacatgaacatggtgtaccgcttattgtagataatacattccctactccggttaatttaagacctatagaatggggagctgatattgttacacattctacaacaaaatatatggacggacatggtgcagctgtaggcggtgcgattgtagacagtggtaagtttgactggatggcacatgctgacaaatttccgggactttgtactcctgatgagtcatatcatggaattacatatgcagagaagtttggtaaagagggtgcgtttataactaagtgtacttcacagcttatgagggatttgggctgcattcagtcacctcagaatgcttttattttaaatctcggtctggagtcacttcatgtgcgtatgccaagacatgtggagaacggacaggctgttgcagaatttcttgagaagcaggataaggttgaatttgtcaattatccgggacttccatcgaataaatattatgaaatagcaaagaaatatatgccaaacggaggttgtggtgtagtttcctttgaattaaaaggtggaagagaagcagcagaaaagttcatgaagaatcttaaacttgcagcaattgagactcatgtagcagatgcgagaacatgctgcctcaatcctgcaacatcaacacacagacagatgaatgatgaacagcttgcagcggcgggaattccggctggacttatacgtatatcatgtggtcttgaagataagaatgatttgattgccgatatcaagcaggcacttgatgctatttaataaaaatttaataaacatcttcaaataagcttgaagagcatgttaattaagtaatatttcaaaaggagaagctcacgggcttctcctttttgaactcaggttattcattatattaaaatcttgcaagcatacggcttaggattttatcgcatataaatgtataagcagaactaaagatatttgcacttattcttaaaataacagcgtattgggcaccgaaaagccctgctgcaatagatagtgcaccaaatgtattaaaagctgtctgccatgcatatcctgctgaatatccaacgccgataccaccgattattcctatataagggtacattgatgaaggcaggatatagtaaaggacaaggaaaagaaggcatccgactatattaaaaagtcctcttttcacagctcttggctttatgtcatttgaaaatggaaggcagactgacatacatgcgatacctgcccacatggcacgagggaggttgagcagtgacattgcaagtattgaacttgatacaataaaagttagtttaatataccattttgtgcgtgatgaagtaatattaaattctgaaaataaatttttaaaagtacgatggtaaggtctgtttttctgatttttgtaaaaaacaatcatgcatataagcatgcctgctgacaaacctgcaattcgcataagataatcgtgtccgcttacatcatatcccagaagcaggagataactgagaacaaatgttgaatggttatacataatgacattatggcagctgaaaatcataatgagcattatacataagaaattaataaaaaatgccggtatggcaggcaccatatttgcaagacgaggaccggcagctaatattacaaatatacctgcaattgaaataagtccgtgatttgtctgaataccaaaatcagcctgtctgagtacaagtacagaaagcagaacgcatacaccaacgacgctgttactgtttcctgtcaacatactgaaaatagttacaattgcaacgcagaatatcattacaagataaaccttgaaattatatattaaaatatgtctgaatttttcttttttgtcatgtgtatttttgattaactgcttagaacctgcagaactaagctgtaattcctgataaaatgtcatagaaatctccttgtgatgatgtaaaaataatccccacaatagatagggttgaataatataacattaatgacggattatgtcaaataattttaattttacatgtaaaattttagatgcaaatttaaataataagtaccaatgcaaaaataaaagttatataataaaattatagggaagaaggggaatgacagctgcctgacaaaaaagggagagagtatcaggacagctatcggagtttatgaaaaggttttatctaatgtctttcgacattgtctatattacagataaaatgtgattaataattgttgattttatgaaaaaattgtgaaaaacatgaagtgtctcgaagaaaacaaaaaacaggatacagaaaggcggttaatatgaaagttggaattataggaatgggaaatatgggtgcaaaatatgccctgatgatagcaaaaggtgatgtcaaaggcatggaacttgctgcgataacaagagttactgatgaaagatgggatatgattagacaatatgtttcacctgaacttatgaagtttaattcgggtgacgatatgtatgccgccatagatgacaacagtttgtcagtggatgcagtaataatagtaacaccacattattcacatgaagaacttgttaagaaagcgtttgaacgtggaataagtgtactttgtgataaacctgcaggagtttattcaagacaggcaagaagtatgtgggaggcatatcagagtgcaaaaaaaagctgtccgaaacttcaatacggattcattttccatcagagaacatttcctgtttatagaaaaataaaagaaataatcgataataaaacgtatggtaatattaaaagggtaaactgggttattacagactggtacagacctgatgcatattatgaatctggtaaatggagagcaacgtggaaatatgacggaggaggaactctgcttaaccagtgtcctcataatcttgatttattgtcatggatatgtggaaatccttcagatgttgccggattttgcagcgaaggcaagtatcatccgatagaggtagaagatgatgtcacggcatatatggaatgggagaatggtgcaagcggagtgttcattgcatcaacaggcgaggcatccggtgtcaacaggctggagataagtcttgataatgctcttcttgtatgtgacaaaggccagcttcgtgtatgtgaacttgacaagccggaaatagaatacagaagaggaaatggtgatttatttgctaagccaaagtatgagtggaaagatataaagacagagccaaatgatggtgcatatgaaaaactactgacagaatatgcagcgggaaggtgtgttgcaaatggagacgaagctattaacagtctttatttatccaatgcgatatacctttcatcatggaaaaacagaaaagttgcattgcctgtaagtggcagcagatatgaacttatgtttgaaaaacagtttgaaaaagaacttgaaaaaaagattaataaagctataagacgtaagtgatagaatagcagaagccgcggttttatataactgcagcttctaaagatttttcgagatttttcaattcttttaattctcttattgtaaaatatccacatacaatggcggcggctgcatcggcgataggacctgcatacattacgccatcaattcccataaaaatagggaaaataattataagcggaagcaggaacaatatctgtcttgtaagtgacataaatacaccaagtcttgatttacctatagaattaaagaaatttgatgttacaggctgtattccgttgatgattgtcaaaaacatatatattctgaaatacctttcagaaaacttaaaatataattcacttccgttaccgaatatactgataatctggcgtggaaatacctgaaaacatacaaatgcaataagcgataaaccggaagctgcagcaagtgccagcagatatgttttacgtacacgtttatatttttttgctccgtaattaaagccgataacaggctgtgttccctgagatattccgattacaaatgacataaatatcatatttacttttgtgataattccggcacatgcgagaggaatatcaccgccatatacagacattgcaccataatgtgcaagtgtgttattcattacaatctgaacgactgtcattgcaatctggttgaaacatgcacttgctccgaggataaaaatatttttccaatgatgcaatgaaactgtaaagctgcttttgcttaattttatatttttaaaatgaaacatatagcgtaaacttatgcaaaatgaaattatctgtccaataatagttgcaagtgcggcacctgtcattcccatcttaaatacaaatatgaagacaggatcaagaattgtatttacaattgcaccgataagcattgatgtcattgaatagtttggactaccatccgcgagaatcagcttgctcattcctgtattggctatcagaaatggaaagcctatagcagtaatacgcgtatatgcctttgcatatggaagtacatcaggtgttgcaccgaaaaatttaagcattggcgtaaggaatatggtggttataagaaataaaaatattcccattatagccataagaattattccattgccggcatatttttctgattctttcttttcacctgcaccaagatgcagtgagtaattggttgcacaaccaattccgagcaggagtgatatagccgtgcatgttgttgatagtggaaatgcaatatttgttgctgcatttccaagcataccgataccctgaccgataaaaatctgatcgacgatgttgtataacgcagtaacaagcattgatataatacatggaattgcaaatcctacaagaagttttccctcgtttttgtacccgagtggattttcattttcattattgatgctcattgtgtgttatcctttctgataaaataatcaattattcttcaaatgaaacaatctgtgttgcattattataacccagctgctgcatttttttctggagattattaagagctttatcatattttaagaaagcatcatatgtatcgttaaaatatgagagagcttttttgtcgtccccatcttcttttgcctgccatgatagagtggcaaagtgatgaagaacttcatgtgttttgacaagctgaccaaattccggactttcagtaagacggctgtcagtctggctgttcagccattttccaagcttgcatcccaaagggttatcaacctgttttttctttaagtgttcaaatccgacaatattgttgtatacacgccatgtaagaacatagtgatctacttcaaatacacgcatccagtcctgctgtgtgatattagaacatccacgaaccaggtcacttctggtcttgtcaagataacgtcctaatttaaatacacgttgtcctgattgtatacattcatcagataaaatgttataactttgagaaatattttcaatttgttttgctaatgattttgttacaccggtctgcgtgtcaatgtcgttaaatatactgtcaacgcttacgcttatgttgtccatctggccattcatctgctgtatggcggcaagcgactgctcaactttcttatttccttctccaagtgtgagagttgtctcatccattgcagaggcaagtgagtcaatgctttctcttaattcagcaacatattttacaatatcttcggctgattgtgaagtgtttgaagataaaattcttacctgttcggcaacgacagaaaagccttttccggcttcacctgctcttgcagcttcaatagaagcatttaaagcaagaagattactctggcttgcgacattttggacaatgtcaacaatttcttctattttattgatttttcctttgaaatcctggacattctgattgatagtattgattttttcagatgatttattgacagctttaatactgtcattcatgtctgtagttatattttgtgaaactgatagaattttgtgagtgtttgtgcggatatctcccattgcatcagaaatgttttcaatagaactttcgagatgctggctggaattttccatgtgctgtattgattttgtctgtgattcgacctgttccagagtattctttataagagtgttgtcaccgataacacccattgtttcattgagacgcattacaacagggttatttacttctgtaaaaacatgcagcattttattgagtttttcaccatattcggggttgttaaagtcaacaggattaacaccgctgaagttacctgcaataattttatcaatagtatcaagtatctgtaccgcatcatggtcagacaaaatctcatccttttttttccttttaaacat

SEQ ID NO: 8 - DNA, 6247 bp, unidentified
  source 1..6247: mol_type = unassigned DNA
atgaatatgacaacaaatgtaattaacaagggttggttcaatcccatatccctttccgtattgctgatactggttatcgtatcagccgttttgtatgtttcgaagattataaacataccgatattcatcctcttgcttcttctttccgggttggtgacttctgcaatccgcatagccgaccaatgggaacgagcggtagtccttcgtatggggaaatacaacggtttgaaaggtcccggcccctttatgattatcccggttatagacagtgtatcgacttatattgaccagcgtgtacgggtaagtgcatttaaggccgaacaaactctaaccaaagatacggttccggtaaatgtggatgcagtcgtatattggaccgtctgggacgtggaaaaagctgcattggaagtacaggagtaccaaaaagcgatcgaacatatcacacagactggtctaagagataccatcggtaagcacgaactgtcggatctcttgcaggaacgcgataagatagctgaggatttgcaacaggtcttagacagaaatacgaatccgtggggcatcacatgccagactgtcggtatcaaagatatcgccattccgcaagacctggccgaagcaatgagcaaagaggcccaggccgaacgtgaaagaagagctcgcgtcatcttgggaacagcagaaacggaaatcgcagaaaagttcgaacaagccagtaagaaatataccgacaatcctgtggcattgcatcttagagggatgaatatgctgttcgaaggcttaaaagaaaaaggatcgatggttatcgtaccgagttcggcactcgacaccatgaacttaggggctatgggaggcttggtttcattggctaagaacaacgagacaccgaaataaactattttctaacaacaaaaaatcaccatttatggaaatacacaaacaagggattacttttgtactccttctattaatctttacaaattgttcacgaaaaccatcgctgcaatggattcccttcagttgggaaggtgatacaatttcggggatatacattgaaaaagcttttctaaatgttccggtaaaaatagagaatttaccctacgaattcacaatgcagtttgatttaggagcatataattctgtcttttacgggaatacttttgctccttatctcaaagaagccccatctttaatgaacaaaaaagattcaaccgggatgtataagaatgtcaacctgcaaatagggacagtggagtttagcaatgccaatatcggattcatgcagaatttcggaaataaaatccctaaagattcacttcattcaaatacccccaagcatattggaacgatcgcatccgacatggtgcaagataaagtcttaatcattgattacaaatcaaacaggcttgcaatcacagactttttaccggctgaatacgaaaatctgctggctgaagaatttgaatcagaatatggggaaataaaattgcctttacggattaatggaaaaacatgtaaagtattgttcgatacaggttcaagccctttccagttaataacgacacaagaaagagcttggagcgtatcagaccttactattgtcgactctctaagcggtcctttatggtggggacaagaaataacattttatggctttaatgtaaccaaaccgatcgagttcggcggaaaagccttgaacaattcgaaagtctactacgccaaagacggcctttgggatagtatttatagtaccctcgatgtctggggaataaccggaaatgcttatttctttgaaaacaccgtgattatcgattacaaaaacaaactattcaggataaaataaaaattcgaacccatacttcttcattcagatgcaagcgtaagttgttttctacattgccgtatatattcccgacaacgtcaaatatattctcgacaacgtcgaaaacaacttacacttgcatcaaaacaaaaacacattgccatcttacatcaaacctttgccagagcttggtctaggtcatcaatgatatcatcgatatattcagtacctatactcaggcgcacagtggaaggagtgatatgctgtccggccaactcttccggagtcatttgagaatgggtagtcgtatacggatggatcaccaggctcttcacatcagccacattggcaagaagggagaatatttccagcgaatcgataaacttccaggccgcttcctgcccgccttcgatctcgaaagtgaaaatacttccggcacctttcgggaaatactttttataaagttcatggtctggatggtctggaagagccggatggttcactttggttactttcgggtgtttggataaatactcgacgactttcagagcattggcaacatgacgttccacgcggagagacaatgtttccagaccttgcagcaggataaaagcattgaaaggcgagatcgtagctcctgtatcacgcagaatgacagcccggatacgagtgacgaaagctgtcggacccgccacatcggcaaagaccgccccatgataacttgggtccggtttgcccaaggtcgggaacttatcggcatttgctttccaatcgaaattgccactgtccacaatcactcctcccaagctcgatccatgtccgccgataaatttggtggcggaatgaacaacaatatcggccccgtgctcgatcggacggatcaggtacggtgtaccgaacgtattatccactatcaacggaatattatgacgatgggccacctccgccacagcatcgatattggtaacatcagaattaggattaccgaacgtttcgacatacaccgctttcgtattctcccggatagcagcctccaacgcctgcaagtcattgacatttacaatcgtgtgggtaatgccctgagtcgctaacgtatgcgtaatcagattgaaggaaccgccatagagattgtcggcagccacaatatgatcactcactccaagtatattctgcaaggcatacgtcacggccgccgcaccggaagccaccgccagaccagccactcccccttcgagagcggcaacacgcgcttcgaaaactccctgggtcgaattggtcaggcggccataaatattcccggcatcacgaaggccgaaacggtctgccgcatgttgtgaatcatgaaatacataagatgttgtctgataaatagggacggcacgagcatctgttgcgggatcggcctgttcttgccctacatgaacctgtaatgtctcgaaatgtaatttctttgttaccatagtcgtattattttaaattgttcttcttgctcattttccagcaaaactatagcagacagagatatcagacaagatatatggaaaatacagaaaataacgctatttttacggtcgtcaacagaacgcataaactgtatcaggatcgaaaaaaggattctgatagaattttattcttatgaacacacttgagaaattagacaaagtagacttgcagatcctccgtaccctgcaatcgaatgcccggctcaccacaaaagagctcgccgcgagtgtcagcctctcgtcgacacccgtattcgaacgcctgaaacgcctcgaaaacggaggatatataaagaagtacattgccgtgctggacgccgaaaagctaaaccagggcttcgtggtcttttgcagtgtcaagttgaggagactgaacaaagagattgcggcggagttcacaagaatcatacaggaaattccgcaagtcacagaatgctacaatatatcgggaagttacgactacctgctgaagatccatgcaccgaatatgaagtattaccaggagtttatcttgaacgtgttaggcacgatcgacagcctcggatcgctcgaaagcacctttgtgatggacgaggtaaagcatgaatacggaatccatatttaaacgccgccccgtccctatcttacatttattacttcaatgcataaatccgctggatgatgtccacaaccttcatgccttccaatacgttggtcgtaatacattccgaacttgcaccggaaaggacacggaccacattgcggatcacgaaattatggttctgggcggaacctttataagggccgtagtcatttcccggattcgtcggcgccagttccggcatttcataatctttgatatggcagtattcgacttcgttcatatactgtccgccgattttcacgcttccgttctccgcaacaatcagcatactgctttccatgttcctgttccagacagaagtggaatagctcaggctccccatcccgccgttcacgaagttgaaattcacaaagcccgaatcttcaaaagccgtaagtccggcatggttgaaatcggcaaagcgagcctggatattgcagatatccccgaaaagccagtacataatgtcgataaaatgggagaactgggtaaaaagcgtccctccgtccaaagcggcatcgccatgccagccaccgggtttataatagcgctcgtcacggttccaataacaattcaactggaccatatagatcttgcctaacctacccgaatccaccatctccttgatccagacggaaggaggagaataacggttttgcatgacgcagaacacctgtttacggtatttcagcgaagcatagaccaccttctcggcatcggcaagcgtcagtgccatcggcttctcgatcacgacgttgcaaccggtttcgacagcctgaatggccattgctgcatgcaagccgttcggggtacaaacatttacgacatctatggaaattccgctctgcaatagctccgtcagggatgaaaaaaacgggacatcgtatgcctcaatacccaattcctcacagggacgaatatcacaaagggccaccaattcggctcccggatcgcgtgtcaccatctcggcatgacgtttgccgatatggccacaaccgataactgcaaattttatctttttatccataagcattcgatggggggggtgtcaaaatctccttgtccccgtgcttgacgcggggccgcaaaaggacagaccttatcaatcagatttttatagggtcggttctaatgcgatcccacgtcaagcgcgggaacaggatagtttcgacacacccccttttttagagtttctgttatatattgctgttgtccgagggacatttccgtatgaataggcagggaaaggacggattcgcacaaacgggtggagacgtccaaattgccggatacacgggagatatgccggtaagcttcctgatgatgcactggtacaggataatagatcatggaagggattccctgttctttcaacaggcattgaaggacattccgcctgccgtcttttacctggaccgtatattggtgatagacatgagtcgaaaatatcgatctcgcagggagaaccaacgaatcgcaggaggacaaagcttcatcataccgagaggcaacttgccgacgggtttccgtgaactcctccagatgcttcaacttcacatccagcacagcagcttgcaacgtgtccagacgggagttgcatccgatccgcctatggtggtatttgcattcctggccatgattggcaatcatgcggatacgggcagccaacgcctcgtcggaagtcatcaacgcccccccatcgccatagcaggccaacggcttggatggaaaaaaagatgtcgtcccgatatgtccgatcgtcccggcctttcgtacccgcccgtcggaaaagatataatcggcaccaacagattgagcgttatcttccactacatacagatgatagacatctgccaaagtcatgatcggctccatatcgcaggcttgtccgaacaggtggacggccacgaccgcctttgtccgttccgaaacagccgcttctattttccggggatcgatattaaaggtacagggatcgacatccaccaacacgggcgtcaaccctaacaaagcgacaacctcggctgccgctatataagtaaatgccggcagaatcacctcatcgccaggctgaagctccaatgccatcagagcaatctgcaaagcgtccgtcccgttagcacaaggaatcacgtatggaacctgaagataatcagccaaatgcccggcaaaggatttcacctgcggaccattgataaaagcacaactgtcgatcaccgcctgcatggcctcatcaatctcctctttgaaccggagatattgtccatgcaagtcgaccatctgtatctgttcttccaacat

SEQ ID NO: 9 - DNA, 5467 bp, unidentified
  source 1..5467: mol_type = unassigned DNA
tattcacccactgaatccgaagttatgatgaacgagaacaacgatgtttttacaatggaagacgagccgatagcctctgttatgcagaatatgcgcaaaggctcgaaatggctgtccgcatttctggaaagttaccgtcctccgttggacggggagcgttacctgacggacggcgaggtgtcggaactgctccgtgtgagccgacgcaccttgcaagaataccgcaacaaccgcgtgttgccctttatccttttgggagggaaggtgctttacccggaaacggtgctgcgcgaggtgctggaagcgaactaccgcaagccgttatgatgttgattttgtagttagaaataaaaagattacttttgcaatctgtttacgcttagtcgtaatcacttgctcaatctcatgtgcgcttgggtttccaatgccatcgtacttccagctcgttctgggagcagtttcattctttaatacgttgggaaaggggggctttaagtatgacaaaaactataatgaaggcggtaggggctgtagcggcaggccgaaacaggaaagttcatatgaggatagcaagtgtaaccatatattgcgatgaaggctccaagggttcgaatcctgccaccccgacaaaagccactgataatcagtggctttttcttttttcggacacatttcggacaaattatatctgaatattgattattcaataacctaagcaaagttaagttaagctagaagaataacccttcttaaagaatgattggaacataatgcttgtttctatagagcgtgaaaagttcatcttattcttataaaaaatcaagagggctaggagtcttttgcttgtctatgtcagtgatatgagtgtagatcatagtcgtttttatattttcatgacccaataactcttttacaattcggatatcgatacctgattggattaaatgtgtggcataagaatgtcgcccagtatgtacagagccttccgactgaacatttgctttgatcaatgcttttttcagaatcaactgtacacttcgttcgctatacttgccacccttctcaccttcgaagaggtactcctttggcttgaatgcttgataataatgtctcaaagtcgccagcaatttgtctggtaatgatacaatccgatctttattacctttgctttgatggattctgataattccatcggaagattttatgtctttgattttgagattcaacagttcgctaagacgtagtccacaactatagattgtcatcagaatagccttgtgttttagattttgagtgttatcgagaatatttcttacttcttcttttgaaaagaatttaggtaatttagaaaatgagcgtttcgggtataagtaatccagttggatttttttgtccaatatcagttcgtacatcttcttgattgcgcccactaatcctttttgataggaaacactaattttcccattttgaactttttcgtttataaacgcctcaatatctgacagtggaatttcttcaagggagggatatttactaacatgtttaagaaatatactagcgtaatccttgtacgatctaatggtattgccagcatatctctgcatcagcaatctcttttcgaaggtttcaagtatgatttcgctattcatatcagggaaatagtattttataatattttgtttcgctatttgtgcggttttattttttaaatacaaatatatcaatatcttatgtagaaagaaaagtcaaataacgaaacacgtatatagggttggttaagcgaaattttccgtatattcgtttcgttaaaacagatgttgttatgcggcagcttaaaagacgacaccaaaccagaaaaatcatcttgacaaccacccgactttgaactacgaaggatgaaatttttcagggacaacttccagcatttccaaaaaacagtttaaccattgacttggagacaaacaaacaatttgagcattaaggtttaaaccgaatttttccgaaattgtcctgacctgacttttcctgaaaatcgactttaaagctgtttttacagataaatcaggtttctctaacagacaggaaataaatgctaagtatttggctttaatcttaaaatcaaaaaataagtgttttcttttaatgtttaacagggctgattttacagttggcggtggcaagaaactttcaggacctacctcatagacaagtttcaaatcaaaaaaagtatgatagaaaacggtatatggattgtaaagcttccttgaaaataacttttatgtaggttcgaactgaaggacaatggaacctcccagaaaattttcaagattctcaaacatcaggattttgaaaatatcggaagtaatgccataaggaatatttgacaccactttgaaaggaaatttcggaactgcaaaattcctaaaatcacaaccgacaacttgaacatttcgggcatcagaaaataattttcgtaaatgttcaaccaaagctgtgtcgttttcaatagcaacaacattgttggcaatttttaataaatgaacagtaagaaaccccttgcctgccccaatatctaaaaccgtatcctgattacttatatttgcttgtcttattgcatcttttattagcactttatcaatagtaaagtgctgacccgtaaaacgaacgggcaatttcttttttgtcattagtaacttcttacaggtgaatacttgaattcaaatcacagagatattcttttataattatacaacggcaaatttagtaactttgctcgataaattaacgagaaagaagaaaaaatgaaagccgacagcataacatcacctatacgcaagcaggggtttcgtgcttctatgacagtgaagtggtaaatttgaagttcagttcttcgtaggaagttcagtggttaaaatccctgcctgcgtatagctgagaaccgttgtgggctattttgcacacgacacgacaaacatcaaatgactatcgaaatatgagtttattaaacaataaaaaagttgcaattatcggtggcggaccagttggactaacaatggcaaacttgttacaacaaaacggtattgacattaccgtttacgaaagagatgaaaacccaaaagcacgagtttggggcggaacgcttgaccttcacaaaaattcaggacaagaagcaatgaaaaaagtaggattgttgcaaacctactatgatttggcgctacctatgggcgtaaactttgctgatgagaagggtaacattatagcaacaagaaacccgacactcgaaaataagtttgacaaccccgaaataaatagaaacgcgttgcgaaaaatgttgcttggcagcttgaaaaatgacacagttgtttgggatagaaaatctattgggcttgaacaagaaaacggaaaatggctgctacattttgaaaataagccaactgcattggccgactttattattgtttccaatggtggaatgtctaaaataagaaattttgtttcagataatgaagtcgaagaaacaggtacttttattattcagggcgacattcctgaaccagaaacgaactgccctgaattttataagttgtgcaacaacaatagactaatgaccgcacatcaagggaatttattagttgcgaatccatttaacaacggaatgttaacttacggtgtcattttcaaaaagcctgaagaatggaataatggaaaaggattagattttaaggacacaaaaagcgtttccgagttcttgacaaacaggttttcaaattggagcaatgaatacaaggagttaattcgttcaacaacttttttcgttggtttaacaataaaaatatttccgctagacaaaaagccttggaagagtaatcgtccgttacccataactttaattggcgacacagctcacctaatgccaccttttgcagggcagggcgtaaacattggactaatggacgctttgattttgtcagaaaatcttacaaacgggaaatttggaacgatacaaagtgctattgatgactatgaacaacgaatgtttgtttacgcaacagaagcacaagcggactcgacaaagaatgaaatagaaatgcgaaatccgagctttacttttcaacagctaatgaatgtataactcgacagacaaaaaaaacagcccacaacaatcggcttgaaacgcaaggcggggtaaacgtcttcgattgaacatttttttctaattttacggtatcggcttcgattgaaaggtagtggtaaaaacccgccctgcgtcaagccgccggacgttatgtgccatttttgaatgacaatgcgaatagatacagacaaacaaatgaatttacttagtgataagaacgttgcaataattggtggtggacccgttggactgactatggcaaaattattacagcaaaacggcatagacgtttcagtttacgaaagagacaacgaccgagaggcaagaatttttggtggaacccttgacctacacaaaggttcaggtcaggaagcaatgaaaaaagcgggattgttacaaacttattatgacttagccttaccaatgggtgtaaatattgctgatgaaaaaggcaatattttatccacaaaaaatgtaaagcccgaaaatcgatttgacaatcctgaaataaacagaaatgacttaagggctatcttgttgaatagtttagaaaacgacacggttatttgggatagaaaacttgttatgcttgaacctggtaagaagaagtggacactaacttttgagaataaaccgagtgaaacagcagatttggttattcttgccaatggcgggatgtccaaggtaagaaaatttgttaccgacacggaagttgaagaaacaggtactttcaatatacaagccgatattcatcaaccagagataaactgtcctggattttttcagctatgcaatggaaaccggctaatggcatctcaccaaggtaatttattatttgctaaccccaataataatggtgcattgcattttggaataagttttaaaacacctgatgaatggaaaaaccaaacgcaggtagattttcaaaacagaaatagtgtcgttgattttcttctgaaagaattttccgattgggacgaacgctacaaagaattgattcatacgacgttgtcatttgtaggattggctacacggatatttcctttagaaaagccttggaaaagcaagcgcccattacccataacaatgattggggatgccgcacatttgatgccgccttttgcagggcagggagtaaatagtgggttggtggatgccttgatattgtctgataatctagccgatggaaaatttaatagcattgaagaggctgttaaaaattatgaacagcaaatgtttatctatggcaaagaagcacaagaagaatcaactcaaaacgaaattgaaatgtttaaacccgactttacgtttcagcaattgttaaatgtataa

(19) United States
(12) Patent Application Publication
(10) Pub. No.: US 2024/0392375 A1
(43) Pub. Date: Nov. 28, 2024

(54) DIAGNOSIS AND TREATMENT OF DISEASES AND CONDITIONS OF THE INTESTINAL TRACT

(71) Applicant: FLAGSHIP PIONEERING INNOVATIONS VI, LLC, Cambridge, MA (US)

(72) Inventors: Douglas Gowers COLE, Wayland, MA (US); Jason PARK, West Newton, MA (US); Sabrina Yusang YANG, Cambridge, MA (US); Boris HAYETE, Needham, MA (US); Andrew John GASPARRINI, Boston, MA (US)

(21) Appl. No.: 18/694,568

(22) PCT Filed: Sep. 23, 2022
(86) PCT No.: PCT/US2022/076932

§ 371 Date: Mar. 22, 2024

Related U.S. Application Data
(60) Provisional application No. 63/247,458, filed on Sep. 23, 2021.

(51) Int. Cl. C12Q 1/6883

(52) U.S. Cl. CPC C12Q 1/6883; C12Q 2600/118; C12Q 2600/158

(57) ABSTRACT

The invention relates to diagnostic and therapeutic methods for inflammatory bowel disease (IBD).

### SEQUENCE LISTING

The instant application contains a Sequence Listing which has been submitted electronically in XML format and is hereby incorporated by reference in its entirety. Said XML copy, created on Sep. 13, 2022, is named 51373-016WO1_Sequence_Listing_9_13_22.xml and is 113,184 bytes in size.

### FIELD OF THE INVENTION

The invention relates to diagnostic and therapeutic methods for inflammatory bowel disease (IBD).

### BACKGROUND

Mammals are colonized by microorganisms in the gastrointestinal (GI) tract, on the skin, and in other epithelial and tissue niches. The gastrointestinal tract of a healthy individual harbors an abundant and diverse microbial community. It is a complex system, providing an environment or niche for a community of many different species or organisms, including diverse strains of bacteria. Hundreds of different species may form a commensal community in the GI tract in a healthy person, and this complement of organisms evolves from the time of birth and is believed to form a functionally mature microbial population by about 3 years of age. Interactions between microbial strains in these populations and between microorganisms and the host, e.g., interactions with the host's immune system, shape the community structure, with availability of and competition for resources affecting the distribution of microorganisms.

A healthy microbiome may provide a subject with multiple benefits, including colonization resistance to a broad spectrum of pathogens, essential nutrient biosynthesis and absorption, and immune stimulation that plays a role in maintaining a healthy gut epithelium and appropriately controlled systemic immunity. Conversely, an unhealthy (e.g., dysregulated) microbiome may be associated with a disease state.

There is a need for methods for addressing problems in healthcare through assessment of the microbiome.

### SUMMARY OF THE INVENTION

In one aspect, the disclosure features a method of diagnosing inflammatory bowel disease (IBD) in a subject, the method comprising determining a level of one or more of SEQ ID NOs: 1-9 in a sample from the subject, wherein a level of one or more of SEQ ID NOs: 1-9 that is changed relative to a respective reference level for SEQ ID NOs: 1-9 indicates that the subject is likely to have an IBD.

In another aspect, the disclosure features a method for determining an increased risk of an IBD in a subject, comprising the steps of (a) measuring the nucleic acid level of one or more of SEQ ID NOs: 1-9 in a sample collected from the subject using amplification and one or more pairs of primers specific for each of the one or more of SEQ ID NOs: 1-9; and (b) using the amplification results to determine whether the level of one or more of SEQ ID NOs: 1-9 is changed relative to a respective reference level for SEQ ID NOs: 1-9, thereby determining an increased risk of an IBD for the subject.

In some embodiments, the method comprises determining or measuring a level of at least two, at least three, at least four, at least five, at least six, at least seven, or at least eight of SEQ ID NOs: 1-9 in the sample from the subject.

In some embodiments, the method comprises determining or measuring a level of all nine of SEQ ID NOs: 1-9 in the sample from the subject.

In some embodiments, a level of one or more of SEQ ID NOs: 1-9 is changed relative to the respective reference level for SEQ ID NOs: 1-9 in the sample from the subject and the method further comprises administering an anti-IBD therapy to the subject.

In some embodiments, the anti-IBD therapy comprises an anti-integrin therapy. In some embodiments, the anti-integrin therapy targets integrin α₄β₇. In some embodiments, the anti-integrin therapy is vedolizumab.

In some embodiments, the anti-IBD therapy comprises a biologic therapy, an anti-inflammatory agent, an antibiotic, or an immune system suppressor. In some embodiments, the biologic therapy is vedolizumab (ENTYVIO®), infliximab (REMICADE®), adalimumab (HUMIRA®), golimumab (SIMPONI®), certolizumab (CIMZIA®), risankizumab (SKYRIZI®), or ustekinumab (STELARA®); the anti-inflammatory agent is a corticosteroid or an aminosalicylate; the antibiotic is ciprofloxacin (cipro) or metronidazole (Flagyl); or the immune system suppressor is azathioprine (AZASAN®, IMURAN®), mercaptopurine (PURINETHOL®, PURIXAN®), methotrexate (TREXALL®), tofacitinib (XELJANZ®), upadacitinib (RINVOQ®), or ozanimod (ZEPOSIA®).

In some embodiments, the anti-IBD therapy further comprises treatment by fecal microbiota transplant (FMT).

In some embodiments, the reference level is a pre-assigned level.

In some embodiments, the reference level is a level in a set of samples from a reference population. In some embodiments, the reference population is a population of healthy subjects.

In some embodiments, the level of each of SEQ ID NOs: 1-9 that is determined or measured in the sample from the subject is a nucleic acid level.

In some embodiments, the nucleic acid level is a DNA level.

In some embodiments, the change is a decrease relative to the reference level. In some embodiments, the levels of at least 1, 2, 3, 4, 5, 6, 7, 8, or 9 of the sequences for which a level is determined or measured are decreased relative to a respective reference level for the sequence.

In some embodiments, the change is an increase relative to the reference level. In some embodiments, the levels of at least 1, 2, 3, 4, 5, 6, 7, 8, or 9 of the sequences for which a level is determined or measured are increased relative to a respective reference level for the sequence.

In some embodiments, the sample comprises a sample of the microbiota of the subject.

In some embodiments, the sample is a fecal sample.

In another aspect, the disclosure features a kit for diagnosing an inflammatory bowel disease (IBD) in a subject, the kit comprising: (a) polypeptides or polynucleotides capable of determining the level of one or more of SEQ ID NOs: 1-9 in a sample from the subject; and optionally (b) instructions for use of the polypeptides or polynucleotides to determine the level of one or more of SEQ ID NOs: 1-9 in the sample from the subject, wherein a change in the level of one or more of SEQ ID NOs: 1-9 relative to a respective reference level for SEQ ID NOs: 1-9 indicates that the subject is likely to have an IBD.

In some embodiments, the kit comprises polypeptides or polynucleotides capable of determining the level of at least 1, 2, 3, 4, 5, 6, 7, 8, or 9 of SEQ ID NOs: 1-9 in a sample from the subject.

In another aspect, the disclosure features a method of diagnosing an IBD in a subject, the method comprising determining a level of each of SEQ ID NOs: 1-9 in a sample from the subject, wherein a level of at least 50% of SEQ ID NOs: 1-9 that is changed relative to a respective reference level for SEQ ID NOs: 1-9 indicates that the subject is likely to have an IBD.

In some embodiments, a level of at least 50% of SEQ ID NOs: 1-9 is changed relative to a respective reference level for SEQ ID NOs: 1-9 in the sample from the subject and the method further comprises administering an anti-IBD therapy to a subject.

In some embodiments, a level of at least 1, 2, 3, 4, 5, 6, 7, 8, or 9 of SEQ ID NOs: 1-9 that is changed in a sample from the subject relative to a respective reference level for SEQ ID NOs: 1-9 indicates that the subject is likely to have an IBD.

In another aspect, the disclosure features a method of treating a subject having an IBD by a method selected from the group consisting of an anti-integrin therapy, a biologic therapy, an anti-inflammatory agent, an antibiotic, an immune system suppressor, and FMT, wherein the subject was diagnosed as having an IBD by any of the methods provided herein.

In another aspect, the disclosure features a method of treating a subject having an IBD, the method comprising the steps of (a) measuring the nucleic acid level of one or more of SEQ ID NOs: 1-9 in a sample collected from the subject, wherein a level of one or more of SEQ ID NOs: 1-9 that is changed relative to a respective reference level for SEQ ID NOs: 1-9 indicates that the subject is likely to have an IBD; and (b) treating a subject who has been determined to have an increased risk of an IBD with a method selected from the group consisting of an anti-integrin therapy, a biologic therapy, an anti-inflammatory agent, an antibiotic, an immune system suppressor, and FMT.

In some embodiments, the nucleic acid level of the one or more of SEQ ID NOs: 1-9 in the sample collected from the subject are measured using amplification and one or more pairs of primers specific for each of the one or more of SEQ ID NOs: 1-9, and the amplification results are used to determine whether the level of one or more of SEQ ID NOs: 1-9 is changed relative to a respective reference level for SEQ ID NOs: 1-9.

In some embodiments, the method comprises measuring the nucleic acid level of at least two, at least three, at least four, at least five, at least six, at least seven, or at least eight of SEQ ID NOs: 1-9 in the sample from the subject. In some embodiments, the method comprises measuring the nucleic acid level of all nine of SEQ ID NOs: 1-9 in the sample from the subject.

In some embodiments, the reference level is a pre-assigned level. In some embodiments, the reference level is a level in a set of samples from a reference population. In some embodiments, the reference population is a population of healthy subjects.

In some embodiments, the nucleic acid level is a DNA level.

In some embodiments, the change is a decrease relative to the reference level. In some embodiments, the levels of at least 1, 2, 3, 4, 5, 6, 7, 8, or 9 of the sequences for which a level is measured are decreased relative to a respective reference level for the sequence.

In some embodiments, the change is an increase relative to the reference level. In some embodiments, the levels of at least 1, 2, 3, 4, 5, 6, 7, 8, or 9 of the sequences for which a level is measured are increased relative to a respective reference level for the sequence.

In some embodiments, the sample comprises a sample of the microbiota of the subject. In some embodiments, the sample is a fecal sample.

In another aspect, the disclosure features a method of treating a subject having an IBD, the method comprising the steps of (a) measuring the nucleic acid level of each of SEQ ID NOs: 1-9 in a sample collected from the subject, wherein a level of at least 50% of SEQ ID NOs: 1-9 that is changed relative to a respective reference level for SEQ ID NOs: 1-9 indicates that the subject is likely to have an IBD, thereby determining an increased risk of an IBD for the subject; and (b) treating a subject who has been determined to have an increased risk of an IBD with a method selected from the group consisting of an anti-integrin therapy, a biologic therapy, an anti-inflammatory agent, an antibiotic, an immune system suppressor, and FMT.

In some embodiments, the nucleic acid levels of each of SEQ ID NOs: 1-9 in the sample collected from the subject are measured using amplification and one or more pairs of primers specific for each of SEQ ID NOs: 1-9, and the amplification results are used to determine whether the level of each of SEQ ID NOs: 1-9 is changed relative to a respective reference level for SEQ ID NOs: 1-9.

### BRIEF DESCRIPTION OF THE DRAWINGS

FIG. 1 is a graph showing the out-of-cohort performance (as percent specificity and sensitivity) of metagenomic markers for distinguishing inflammatory bowel disease (IBD) patients from control patients. AUC=area under curve.

FIG. 2 is a graph showing the score sum and rank of a model comprising metagenomic markers and a null model.

FIG. 3 is a heat map showing the level (scaled reads per kilobase of transcript per million mapped reads (RPKM)) of metagenomic markers in IBD and control patients.

### DETAILED DESCRIPTION OF THE INVENTION

### Definitions

The term “changed.” as used herein, refers to an observable difference in the level of a marker in a subject (e.g., in a sample from the subject), as determined using techniques and methods known in the art for the measurement of the marker. A marker level that is changed in a subject may result in a difference of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) more or less than a reference level (e.g., a level from a healthy subject or a level prior to treatment) (e.g., up to 100% or up to 100-fold relative to the reference level). In some embodiments, the change is an increase in the level of a marker in a subject. Increasing the marker level in a subject may result in an increase of at least 1% (e.g., at least 5%, 10%, 15%, 20%, 25%, 30%, 35%, 40%, 45%, 50%, 55%, 60%, 65%, 70%, 75%, 80%, 85%, 90%, 95%, 98%, 99%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level). In other embodiments, the change is a decrease the level of a marker in a subject. Decreasing the marker level in a subject may result in a decrease of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level).

In some embodiments, the change in the level of a portion of the markers analyzed is an increase, while the change in the level of another portion of the markers analyzed is a decrease. In some embodiments, the change in at least 1, 2, 3, 4, 5, 6, 7, 8, or 9 of the markers analyzed is an increase relative to a reference level. In some embodiments, the change in at least 1, 2, 3, 4, 5, 6, 7, 8, or 9 of the markers analyzed is a decrease relative to a reference level.

The term “pharmaceutical composition,” as used herein, represents a composition formulated with a pharmaceutically acceptable excipient. For example, a “pharmaceutical composition” can be a composition that is manufactured or sold with the approval of a governmental regulatory agency as part of a therapeutic regimen for the treatment of a disease, disorder, or condition in a mammal, intended for such use, or in development for such use. In some examples, the pharmaceutical composition is a pre-approved composition.

The term “subject,” as used herein, represents a human or non-human animal (e.g., a mammal).

“Treatment” and “treating.” as used herein, refer to the medical management of a subject with the intent to improve, ameliorate, stabilize, prevent, or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

The term “inflammatory bowel disease” or “IBD,” as used herein, refers to a condition of the bowel, e.g., the small intestine, large intestine, mouth, esophagus, stomach, rectum, and/or anus, that is characterized by inflammation. Examples of IBD include ulcerative colitis (UC) and Crohn's disease. Other examples include microscopic colitis (e.g., collagenous colitis or lyphocytic colitis), diversion colitis, Behçet's disease, or indeterminate colitis.

### I. Methods for Diagnosing and Treating Inflammatory Bowel Disease

The invention is based, in part, on the discovery that levels of gut microbiome biomarkers (i.e., markers of bacterial origin), which may be referred to as co-evolved molecules, can be used to identify patients having an inflammatory bowel disease (IBD). Accordingly, the disclosure provides methods of diagnosing, treating, and monitoring subjects (e.g., human patients) based on this discovery.

### Diagnostic Methods and Methods of Treatment

In some aspects, the disclosure features methods of diagnosing inflammatory bowel disease (IBD) in a subject (e.g., a human subject), the methods comprising determining a level of one or more of SEQ ID NOs: 1-9 in a sample from the subject, wherein a level of one or more of SEQ ID NOs: 1-9 that is changed relative to a respective reference level for SEQ ID NOs: 1-9 indicates that the subject is likely to have an IBD (e.g., ulcerative colitis or Crohn's disease). SEQ ID NOs: 1-9 are bacterial sequences.

In some aspects, the disclosure features a method for determining an increased risk of an IBD in a subject, comprising the steps of (a) measuring the nucleic acid level of one or more of SEQ ID NOs: 1-9 in a sample collected from the subject using amplification and one or more pairs of primers specific for each of the one or more of SEQ ID NOs: 1-9; and (b) using the amplification results to determine whether the level of one or more of SEQ ID NOs: 1-9 is changed relative to a respective reference level for SEQ ID NOs: 1-9, thereby determining an increased risk of an IBD for the subject.

In some aspects, the disclosure features a method of treating a subject having an IBD by a method selected from the group consisting of an anti-integrin therapy, a biologic therapy, an anti-inflammatory agent, an antibiotic, an immune system suppressor, and FMT, wherein the subject was diagnosed as having an IBD by any of the methods provided herein.

In some aspects, the disclosure features a method of treating a subject having an IBD, the method comprising the steps of (a) measuring the nucleic acid level of one or more of SEQ ID NOs: 1-9 in a sample collected from the subject, wherein a level of one or more of SEQ ID NOs: 1-9 that is changed relative to a respective reference level for SEQ ID NOs: 1-9 indicates that the subject is likely to have an IBD; and (b) treating a subject who has been determined to have an increased risk of an IBD with a method selected from the group consisting of an anti-integrin therapy, a biologic therapy, an anti-inflammatory agent, an antibiotic, an immune system suppressor, and FMT.

In some aspects, the disclosure features a method of treating a subject having an IBD, the method comprising the steps of (a) measuring the nucleic acid level of each of SEQ ID NOs: 1-9 in a sample collected from the subject, wherein a level of at least 50% of SEQ ID NOs: 1-9 that is changed relative to a respective reference level for SEQ ID NOs: 1-9 indicates that the subject is likely to have an IBD, thereby determining an increased risk of an IBD for the subject; and (b) treating a subject who has been determined to have an increased risk of an IBD with a method selected from the group consisting of an anti-integrin therapy, a biologic therapy, an anti-inflammatory agent, an antibiotic, an immune system suppressor, and FMT.

In some embodiments, the sample comprises a sample of the microbiota (e.g., the gut microbiota) of the subject. In some embodiments, the sample is a fecal sample. In some embodiments, the sample is a colon biopsy.

In some embodiments, the method comprises determining or measuring a level of at least 1, at least 2, at least 3, at least 4, at least 5, at least 6, at least 7, or at least 8 of SEQ ID NOs: 1-9 in a sample from the subject, e.g., comprises determining or measuring a level of 1-2, 2-3, 3-4, 4-5, 5-6, 6-7, or 7-8 of SEQ ID NOs: 1-9 in the sample from the subject. In some embodiments, the method comprises determining or measuring a level of all nine of SEQ ID NOs: 1-9 in a sample from the subject.

In some embodiments, a change in the a level of at least 1, at least 2, at least 3, at least 4, at least 5, at least 6, at least 7, or at least 8 of SEQ ID NOs: 1-9 in a sample from the subject, e.g., a change in the level of 1-2, 2-3, 3-4, 4-5, 5-6, 6-7, or 7-8 of SEQ ID NOs: 1-9 in the sample from the subject relative to a respective reference level for SEQ ID NOs: 1-9 indicates that the subject is likely to have an IBD. In some embodiments, a change in the level of all nine of SEQ ID NOs: 1-9 relative to a respective reference level for SEQ ID NOs: 1-9 indicates that the subject is likely to have an IBD.

In some embodiments, a threshold number of one or more of SEQ ID NOs: 1-9 is changed relative to the respective reference level for SEQ ID NOs: 1-9 in the sample from the subject (e.g., a number of SEQ ID NOs: 1-9 that has been determined to indicate that the patient is likely to have an IBD, e.g., a level of at least 1, at least 2, at least 3, at least 4, at least 5, at least 6, at least 7, or at least 8 of SEQ ID NOs: 1-9 in a sample from the subject (e.g., a level of 1-2, 2-3, 3-4, 4-5, 5-6, 6-7, or 7-8) of SEQ ID NOs: 1-9 is changed relative to the respective reference level for SEQ ID NOs: 1-9 in the sample from the subject and the method further comprises administering an anti-IBD therapy to a subject.

The anti-IBD therapy may be any medicament, treatment, or combination thereof suitable for the treatment of an IBD. In some aspects, the anti-IBD therapy comprises an anti-integrin therapy. In some aspects, the anti-integrin therapy targets integrin α₄β₇. In some aspects, the anti-integrin therapy is vedolizumab. In some aspects, the anti-IBD therapy comprises a biologic therapy (e.g., vedolizumab (ENTYVIO®), infliximab (REMICADE®), adalimumab (HUMIRA®), golimumab (SIMPONI®), certolizumab (CIMZIA®), risankizumab (SKYRIZI®), or ustekinumab (STELARA®)); an anti-inflammatory agent (e.g., a corticosteroid or an aminosalicylate, such as mesalamine (e.g., DELZICOL®, ROWASA®, others), balsalazide (COLAZAL®), or olsalazine (DIPENTUM®)); an antibiotic, e.g., ciprofloxacin (cipro) or metronidazole (Flagyl); or an immune system suppressor (e.g., azathioprine (AZASAN®, IMURAN®), mercaptopurine (PURINETHOL®, PURIXAN®), methotrexate (TREXALL®), tofacitinib (XELJANZ®), upadacitinib (RINVOQ®), or ozanimod (ZEPOSIA®)). In some aspects, the anti-IBD therapy further comprises treatment by fecal microbiota transplant (FMT). Any one or more of these therapies may optionally be used in any of the methods described herein as employing an anti-IBD therapy.

In some embodiments, the respective reference level for SEQ ID NOs: 1-9 is a pre-assigned level of one of SEQ ID NOs: 1-9.

In some embodiments, the respective reference level for SEQ ID NOs: 1-9 is a level in a set of samples from a reference population, e.g., a population of healthy subjects (e.g., a population of subjects not having an IBD and/or a population of subjects having a healthy gut microbiome). In some embodiments, the healthy subjects are healthy human subjects.

In some embodiments, the level of each of SEQ ID NOs: 1-9 that is determined or measured in a sample from the subject is a nucleic acid level, e.g., a DNA level or an RNA level. In some embodiments, the nucleic acid level is a DNA level, which may be detected, e.g., using a PCR-based method. In other embodiments, detection of a level of one or more of SEQ ID NOs: 1-9 can optionally comprise, for example, detection of RNA levels, which can be achieved by, e.g., RT-PCR, RNA-Seq, and/or methods including the use of microarrays, as are known in the art. In other embodiments, the methods can focus on the detection of protein levels, which can be carried out using standard approaches (e.g., immunoassay-based approaches).

In some embodiments, the change in a level of one or more of SEQ ID NOs: 1-9 in the sample from the subject is a decrease relative to the respective reference level for SEQ ID NOs: 1-9 (e.g., a decrease of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, e.g., a decrease of, e.g., at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the respective reference level for SEQ ID NOs: 1-9; or a decrease of 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% relative to the respective reference level for SEQ ID NOs: 1-9).

In some aspects, the levels of at least 1, 2, 3, 4, 5, 6, 7, 8, or 9 of the sequences for which a level is determined or measured in the sample from the subject (i.e., one or more of SEQ ID NOs: 1-9) are decreased relative to a respective reference level for the sequence.

In some embodiments, the change in a level of one or more of SEQ ID NOs: 1-9 in the sample from the subject is an increase relative to the respective reference level for SEQ ID NOs: 1-9 (e.g., an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100%, e.g., an increase of, e.g., 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold, relative to the respective level for SEQ ID NOS: 1-9; or an increase of 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, 95-100%, or more than 100%, relative to the respective reference level for SEQ ID NOs: 1-9).

In some aspects, the levels of at least 1, 2, 3, 4, 5, 6, 7, 8, or 9 of the sequences for which a level is determined or measured in the sample from the subject (i.e., one or more of SEQ ID NOs: 1-9) are increased relative to a respective reference level for the sequence.

In some aspects, a level of at least 1, 2, 3, 4, 5, 6, 7, 8, or 9 of SEQ ID NOs: 1-9 that is decreased in a sample from the subject relative to a respective reference level for SEQ ID NOs: 1-9 indicates that the subject is likely to have an IBD.

Determination of whether a difference detected is significant can be carried out using standard methods, as well as statistical analysis. In some embodiments, a difference detected is a change of at least 5%, 10%, 20%, 30%, 40%, 50%, 75%, 100%, or more, e.g., at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold, relative to a reference level.

### II. Articles of Manufacture and Kits

In another aspect of the invention, an article of manufacture or kit containing materials useful for the diagnosis, prognostic assessment, and/or treatment of individuals is provided.

In one aspect, the disclosure features a kit or article of manufacture for diagnosing inflammatory bowel disease (IBD) in a subject, the kit comprising (a) reagents for determining a level of one or more of SEQ ID NOs: 1-9 in a sample from the subject (e.g., polynucleotides or polypeptides capable of use in determining the level of one or more of SEQ ID NOs: 1-9 in a sample from the subject); and optionally (b) instructions for use of the polynucleotides or polypeptides to determine the level of one or more of SEQ ID NOs: 1-9 in the sample from the subject, wherein a change in the level of one or more of SEQ ID NOs: 1-9 relative to a respective reference level for SEQ ID NOs: 1-9, as described herein, indicates that the subject is likely to have an IBD. In some embodiments, reagents are included for determining a level of at least 1, 2, 3, 4, 5, 6, 7, 8, or 9 of the sequences of SEQ ID NOs: 1-9.

In some aspects, the reagents for determining a level of one or more of SEQ ID NOs: 1-9 in a sample from the subject comprise one or more polynucleotides (e.g., PCR primers) that hybridize to a complement of a locus of one or more of SEQ ID NOs: 1-9 under stringent conditions and may be used to amplify all or a portion of any one or more of SEQ ID NOs: 1-9, as described herein. In some aspects, the instructions indicate that the one or more oligonucleotides (e.g., PCR primers) may be used to evaluate the presence and/or level of one or more of SEQ ID NOs: 1-9 in a sample from the subject and provide instructions for using the polynucleotide(s) for evaluating the presence and/or level of one or more of SEQ ID NOs: 1-9 in the sample.

For polynucleotide-based articles of manufacture or kits, the article of manufacture or kit may include, for example: (1) an oligonucleotide, e.g., a detectably labeled oligonucleotide, which hybridizes to a nucleic acid sequence encoding a protein, (2) a pair of primers useful for amplifying a nucleic acid molecule, or (3) a microarray comprising multiple oligonucleotide probes. For protein-based articles of manufacture or kits, the article of manufacture or kit may include, for example, one or more antibody-based reagents. The article of manufacture or kit can also include, e.g., a buffering agent, a preservative, or a protein-stabilizing agent. The article of manufacture or kit can further include components necessary for detecting the detectable label (e.g., an enzyme or a substrate). The article of manufacture or kit can further include components necessary for analyzing the sequence of a sample (e.g., a restriction enzyme or a buffer). The article of manufacture or kit can also contain a control sample or a series of control samples that can be assayed and compared to the test sample (e.g., a reference sample, as described herein). Each component of the article of manufacture or kit can be enclosed within an individual container and all of the various containers can be within a single package, along with instructions for interpreting the results of the assays performed using the kit.

The following examples are meant to illustrate the invention. They are not meant to limit the invention in any way.

### III. Examples

### Example 1. Identification of Markers

### A. Assembly and Annotation of Metagenomic Markers

Paired end reads from healthy Human Microbiome Project 1 (HMP1; Human Microbiome Project Consortium, Nature, 486 (7402): 207-214, 2012) individuals were downloaded from the National Center for Bioinformatics (NCBI) Short Read Archive (SRA) and assembled using metaSPAdes (cab.spbu.ru/software/spades/). Metagenomic markers were annotated using antiSMASH4.0 (docs.antismash.secondarymetabolites.org/) with the following non-default parameters: -c3--smcogs--disable-embl. Annotated metagenomic markers were clustered using all vs. all diamond (https://github.com/bbuchfink/diamond) blastx. Blastx results were filtered using a python script requiring (a) E-value <1×10⁻⁵, (b) 90% coverage of length of coding sequence, and (c)>50% of coding sequences in a metagenomic marker present, metagenomic markers were then grouped using markov clustering, resulting in a dereplicated library of 8,211 representative metagenomic markers identified in healthy human gut metagenomes.

### B. EMP Selection

A subset of metagenomic markers prevalent in healthy cohorts, referred to as essential microbial products (EMPs), were identified by clustering metagenomic marker presence/absence across 592 healthy patients from various geographic, genetic, and lifestyle backgrounds. Clusters with a mean prevalence >0.7 and z-score >10 within cohort were selected. To take sample imbalance into account, a proportion test was performed to assess stability across cohorts. This resulted in 1321 EMPs. For diagnostic analyses, metagenomic markers were subsetted from the full dataset, resulting in 1171 EMPs. Metagenomic markers were further subsetted, resulting in 590 EMPs.

### C. Quantification of Metagenomic Markers Across Cohorts

To quantify metagenomic marker abundance across metagenomes, raw forward reads were mapped to the library of 8,211 metagenomic markers using diamond as described above. Normalized abundance of metagenomic markers was calculated such that abundance equals number of reads mapping divided by cumulative length of coding sequences in kilobase pairs divided by number of raw reads mapped.

### D. IBD Data

Raw metagenomic reads from several papers (Paramsothy et al., Gastroenterology, 156 (5): 1440-1454.E2, 2019; Ananthakrishnan et al., Cell Host Microbe, 21 (5): 603-610.e3, 2017; Lloyd-Price et al., Nature, 550:61-66, 2017; Li et al., Nature Biotechnology, 32:834-841, 2014; Nielsen et al., Nature Biotechnology, 32:822-828, 2014; Feng et al., Nature Communications, 6: Article Number 6528, 2015; Hannigan et al., mBio, 9 (6): e02248-18, 2018; Qin et al., Nature, 490:55-60, 2012; and Zeller et al., Mol Syst Biol, 10:766, 2014) were downloaded from NCBI, resulting in 454 samples (246 controls and 208 samples from patients having inflammatory bowel disease (IBD)). Metagenomic marker abundance was computed as previously described.

### E. Data Preprocessing

EMP abundance data were transformed using the rank-based inverse normal transformation (Blom), and centered and scaled.

### F. Modeling

For testing predictive performance, logistic regression models of the form diagnosis˜metagenomic markers were fit with BootGlmV2 with nBoot=300 using leave-one-study-out cross validation. BootGlmV2 is an ensemble approach to regularized logistic regression using interactions. Out-of-cohort area under the receiver operating characteristic (ROC) curve was computed using the pROC package in R (cran.r-project.org/web/packages/pROC/PROC.pdf; Robin et al., BMC Bioinformatics, 12:77, 2011), with per-study area under the curve (AUC) values weighted by study size. p-value on the ROC curve was computed using the Wilcoxon rank sum test (blog.revolutionanalytics.com/2017/03/auc-meets-u-stat.html). For feature selection, full models including all samples were fit with (a) actual data and, (b) data with the response variable randomly permuted using BootGlmV2 parameter randomize=TRUE. Features were ranked and considered significant if having a score greater than the maximum score observed in the null (randomly permuted) model.

### G. Visualization

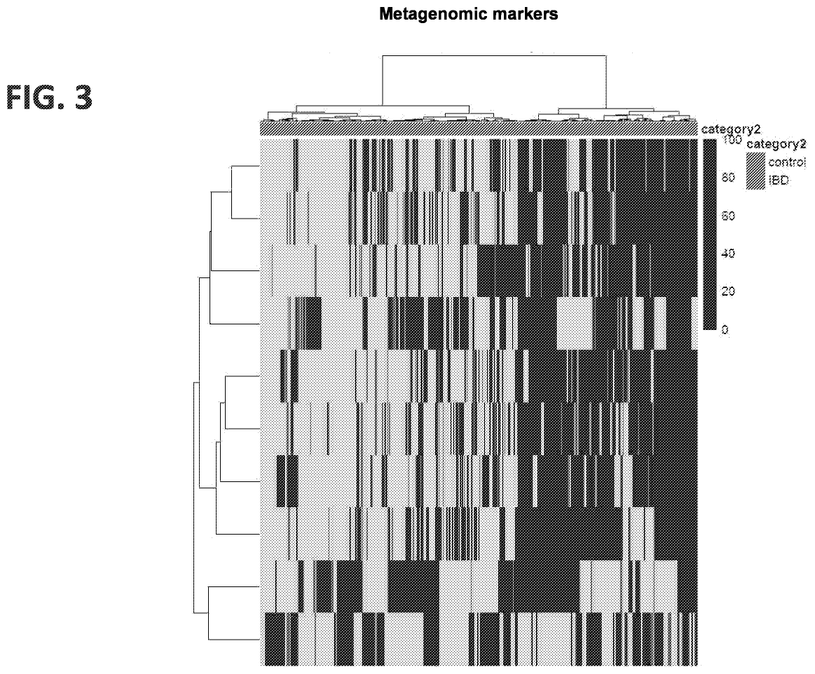

A heatmaps (FIG. 3) were constructed using pheatmap with binarized data and clustering of columns according to Ward's distance.

### H. Conclusions

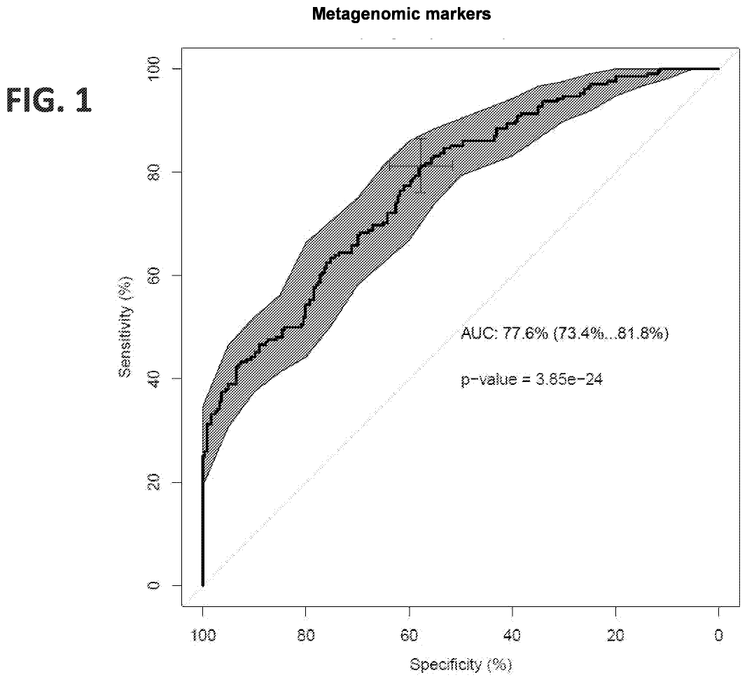

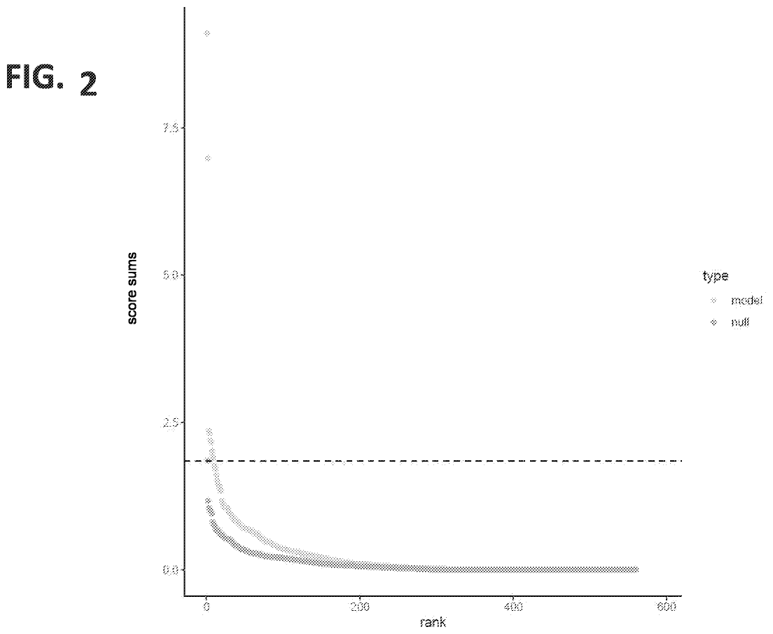

A set of nine metagenomic markers (SEQ ID NOs: 1-9) were found to be significant predictors in a model discriminating between inflammatory bowel disease (IBD) patients and control individuals (FIGS. 1-3).

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of diagnosing inflammatory bowel disease (IBD) in a subject, the method comprising determining a level of one or more of SEQ ID NOs: 1-9 in a sample from the subject, wherein a level of one or more of SEQ ID NOs: 1-9 that is changed relative to a respective reference level for SEQ ID NOs: 1-9 indicates that the subject is likely to have an IBD.

2. A method for determining an increased risk of an IBD in a subject, comprising the steps of:
(a) measuring the nucleic acid level of one or more of SEQ ID NOs: 1-9 in a sample collected from the subject using amplification and one or more pairs of primers specific for each of the one or more of SEQ ID NOs: 1-9; and
(b) using the amplification results to determine whether the level of one or more of SEQ ID NOs: 1-9 is changed relative to a respective reference level for SEQ ID NOs: 1-9, thereby determining an increased risk of an IBD for the subject.

3. The method of claim 1 or 2, wherein the method comprises determining or measuring a level of at least two, at least three, at least four, at least five, at least six, at least seven, or at least eight of SEQ ID NOs: 1-9 in the sample from the subject.

4. The method of claim 3, wherein the method comprises determining or measuring a level of all nine of SEQ ID NOs: 1-9 in the sample from the subject.

5. The method of any one of claims 1-4, wherein a level of one or more of SEQ ID NOs: 1-9 is changed relative to the respective reference level for SEQ ID NOs: 1-9 in the sample from the subject and the method further comprises administering an anti-IBD therapy to the subject.

6. The method of claim 5, wherein the anti-IBD therapy comprises an anti-integrin therapy.

7. The method of claim 6, wherein the anti-integrin therapy targets integrin α₄β₇.

8. The method of claim 6 or 7, wherein the anti-integrin therapy is vedolizumab.

9. The method of claim 5, wherein the anti-IBD therapy comprises a biologic therapy, an anti-inflammatory agent, an antibiotic, or an immune system suppressor.

10. The method of claim 9, wherein the biologic therapy is vedolizumab (ENTYVIO®), infliximab (REMICADE®), adalimumab (HUMIRA®), golimumab (SIMPONI®), certolizumab (CIMZIA®), risankizumab (SKYRIZI®), or ustekinumab (STELARA®); the anti-inflammatory agent is a corticosteroid or an aminosalicylate; the antibiotic is ciprofloxacin (cipro) or metronidazole (Flagyl); or the immune system suppressor is azathioprine (AZASAN®, IMURAN®), mercaptopurine (PURINETHOL®, PURIXAN®), methotrexate (TREXALL®), tofacitinib (XELJANZ®), upadacitinib (RINVOQ®), or ozanimod (ZEPOSIA®).

11. The method of any one of claims 5-10, wherein the anti-IBD therapy further comprises treatment by fecal microbiota transplant (FMT).

12. The method of any one of claims 1-11, wherein the reference level is a pre-assigned level.

13. The method of any one of claims 1-12, wherein the reference level is a level in a set of samples from a reference population.

14. The method of claim 13, wherein the reference population is a population of healthy subjects.

15. The method of any one of claims 1-14, wherein the level of each of SEQ ID NOs: 1-9 that is determined or measured in the sample from the subject is a nucleic acid level.

16. The method of claim 15, wherein the nucleic acid level is a DNA level.

17. The method of any one of claims 1-16, wherein the change is a decrease relative to the reference level.

18. The method of claim 17, wherein the levels of at least 1, 2, 3, 4, 5, 6, 7, 8, or 9 of the sequences for which a level is determined or measured are decreased relative to a respective reference level for the sequence.

19. The method of any one of claims 1-16, wherein the change is an increase relative to the reference level.

20. The method of claim 19, wherein the levels of at least 1, 2, 3, 4, 5, 6, 7, 8, or 9 of the sequences for which a level is determined or measured are increased relative to a respective reference level for the sequence.

21. The method of any one of claims 1-20, wherein the sample comprises a sample of the microbiota of the subject.

22. The method of any one of claims 1-21, wherein the sample is a fecal sample.

23. A kit for diagnosing inflammatory bowel disease (IBD) in a subject, the kit comprising:
(a) polypeptides or polynucleotides capable of determining the level of one or more of SEQ ID NOs: 1-9 in a sample from the subject; and optionally
(b) instructions for use of the polypeptides or polynucleotides to determine the level of one or more of SEQ ID NOs: 1-9 in the sample from the subject, wherein a change in the level of one or more of SEQ ID NOs: 1-9 relative to a respective reference level for SEQ ID NOs: 1-9 indicates that the subject is likely to have an IBD.

24. The kit of claim 23, wherein the kit comprises polypeptides or polynucleotides capable of determining the level of at least 1, 2, 3, 4, 5, 6, 7, 8, or 9 of SEQ ID NOs: 1-9 in a sample from the subject.

25. A method of diagnosing an IBD in a subject, the method comprising determining a level of each of SEQ ID NOs: 1-9 in a sample from the subject, wherein a level of at least 50% of SEQ ID NOs: 1-9 that is changed relative to a respective reference level for SEQ ID NOs: 1-9 indicates that the subject is likely to have an IBD.

26. The method of claim 25, wherein a level of at least 50% of SEQ ID NOs: 1-9 is changed relative to a respective reference level for SEQ ID NOs: 1-9 in the sample from the subject and the method further comprises administering an anti-IBD therapy to a subject.

27. The method of claim 25 or 26, wherein a level of at least 1, 2, 3, 4, 5, 6, 7, 8, or 9 of SEQ ID NOs: 1-9 that is changed in a sample from the subject relative to a respective reference level for SEQ ID NOs: 1-9 indicates that the subject is likely to have an IBD.

28. A method of treating a subject having an IBD by a method selected from the group consisting of an anti-integrin therapy, a biologic therapy, an anti-inflammatory agent, an antibiotic, an immune system suppressor, and FMT, wherein the subject was diagnosed as having an IBD by a method of any one of claims 1-22 and 25-27.

29. A method of treating a subject having an IBD, the method comprising the steps of:
(a) measuring the nucleic acid level of one or more of SEQ ID NOs: 1-9 in a sample collected from the subject, wherein a level of one or more of SEQ ID NOs: 1-9 that is changed relative to a respective reference level for SEQ ID NOs: 1-9 indicates that the subject is likely to have an IBD; and
(b) treating a subject who has been determined to have an increased risk of an IBD with a method selected from the group consisting of an anti-integrin therapy, a biologic therapy, an anti-inflammatory agent, an antibiotic, an immune system suppressor, and FMT.

30. The method of claim 29, wherein the nucleic acid level of the one or more of SEQ ID NOs: 1-9 in the sample collected from the subject are measured using amplification and one or more pairs of primers specific for each of the one or more of SEQ ID NOs: 1-9, and the amplification results are used to determine whether the level of one or more of SEQ ID NOs: 1-9 is changed relative to a respective reference level for SEQ ID NOs: 1-9.

31. The method of claim 29 or 30, wherein the method comprises measuring the nucleic acid level of at least two, at least three, at least four, at least five, at least six, at least seven, or at least eight of SEQ ID NOs: 1-9 in the sample from the subject.

32. The method of claim 31, wherein the method comprises measuring the nucleic acid level of all nine of SEQ ID NOs: 1-9 in the sample from the subject.

33. The method of any one of claims 29-32, wherein the reference level is a pre-assigned level.

34. The method of any one of claims 29-33, wherein the reference level is a level in a set of samples from a reference population.

35. The method of claim 34, wherein the reference population is a population of healthy subjects.

36. The method of any one of claims 29-35, wherein the nucleic acid level is a DNA level.

37. The method of any one of claims 29-36, wherein the change is a decrease relative to the reference level.

38. The method of claim 37, wherein the levels of at least 1, 2, 3, 4, 5, 6, 7, 8, or 9 of the sequences for which a level is measured are decreased relative to a respective reference level for the sequence.

39. The method of any one of claims 29-36, wherein the change is an increase relative to the reference level.

40. The method of claim 39, wherein the levels of at least 1, 2, 3, 4, 5, 6, 7, 8, or 9 of the sequences for which a level is measured are increased relative to a respective reference level for the sequence.

41. The method of any one of claims 29-40, wherein the sample comprises a sample of the microbiota of the subject.

42. The method of any one of claims 29-41, wherein the sample is a fecal sample.

43. A method of treating a subject having an IBD, the method comprising the steps of:
(a) measuring the nucleic acid level of each of SEQ ID NOs: 1-9 in a sample collected from the subject, wherein a level of at least 50% of SEQ ID NOs: 1-9 that is changed relative to a respective reference level for SEQ ID NOs: 1-9 indicates that the subject is likely to have an IBD, thereby determining an increased risk of an IBD for the subject; and
(b) treating a subject who has been determined to have an increased risk of an IBD with a method selected from the group consisting of an anti-integrin therapy, a biologic therapy, an anti-inflammatory agent, an antibiotic, an immune system suppressor, and FMT.

44. The method of claim 43, wherein the nucleic acid levels of each of SEQ ID NOs: 1-9 in the sample collected from the subject are measured using amplification and one or more pairs of primers specific for each of SEQ ID NOs: 1-9, and the amplification results are used to determine whether the level of each of SEQ ID NOs: 1-9 is changed relative to a respective reference level for SEQ ID NOs: 1-9.

45. The method of any one of claims 26, 28, 29, and 43, wherein the anti-IBD therapy comprises an anti-integrin therapy.

46. The method of claim 45, wherein the anti-integrin therapy targets integrin α₄β₇.

47. The method of claim 45 or 46, wherein the anti-integrin therapy is vedolizumab.

48. The method of any one of claims 26, 28, 29, and 43, wherein the anti-IBD therapy comprises a biologic therapy, an anti-inflammatory agent, an antibiotic, or an immune system suppressor.

49. The method of claim 48, wherein the biologic therapy is vedolizumab (ENTYVIO®), infliximab (REMICADE®), adalimumab (HUMIRA®), golimumab (SIMPONI®), certolizumab (CIMZIA®), risankizumab (SKYRIZI®), or ustekinumab (STELARA®); the anti-inflammatory agent is a corticosteroid or an aminosalicylate; the antibiotic is ciprofloxacin (cipro) or metronidazole (Flagyl); or the immune system suppressor is azathioprine (AZASAN®, IMURAN®), mercaptopurine (PURINETHOL®, PURIXAN®), methotrexate (TREXALL®), tofacitinib (XELJANZ®), upadacitinib (RINVOQ®), or ozanimod (ZEPOSIA®).

50. The method of any one of claims 26, 28, 29, 43, and 45 to 49, wherein the anti-IBD therapy further comprises treatment by fecal microbiota transplant (FMT).
